(19) United States
(12) Patent Application Publication
(10) Pub. No.: US 2021/0102957 A1
(43) Pub. Date: Apr. 8, 2021

(54) SYSTEM AND METHOD FOR DETERMINING LUNG HEALTH

(71) Applicant: bioAffinity Technologies, Inc., San Antonio, TX (US)

(72) Inventor: Vivienne I. Rebel, San Antonio, TX (US)

(73) Assignee: bioAffinity Technologies, Inc., San Antonio, TX (US)

(21) Appl. No.: 17/069,272

(22) Filed: Oct. 13, 2020

Related U.S. Application Data
(63) Continuation of application No. PCT/US2019/027550, filed on Apr. 15, 2019.
(60) Provisional application No. 62/657,584, filed on Apr. 13, 2018.

(51) Int. Cl. G01N 33/68

(52) U.S. Cl. CPC G01N 33/6893; G01N 2800/60; G01N 2800/52; G01N 2800/122

(57) ABSTRACT

Predicting the likelihood of lung disease in a subject, comprising labeling an ex-vivo sputum sample from a subject with one or more of the following: a first labeled probe that binds a biomarker expressed on a white blood cell population in the sample; a second labeled probe selected from the group consisting of: a granulocyte probe, a T-cell probe, a B-cell probe, or any combination thereof; a third labeled probe that binds a biomarker on a macrophage cell population; a fourth labeled probe that binds to a disease related cell in the sample; a fifth labeled probe that binds to a biomarker expressed on an epithelial cell population; and a sixth labeled probe that binds to a cell surface biomarker expressed on an epithelial cell population to obtain data comprising a mean fluorescent signature and detecting a profile based upon a presence or absence of labeled probes.

### CROSS-REFERENCE TO RELATED APPLICATIONS

This application is a continuation application of International Patent Application No. PCT/US2019/027550, titled “System and Method for Determining Lung Health”, filed on Apr. 15, 2019, which claims priority to and the benefit of the filing of U.S. Provisional Patent Application No. 62/657,584, titled “System and Method for Determining Lung Health”, filed Apr. 13, 2018, and the specification and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND

Note that the following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

Low-dose computed tomography (LDCT) is the current standard of care for screening for lung cancer as a method of early diagnosis, particularly in high-risk populations defined by the U.S. Centers for Medicare and Medicaid Services (CMS) as individuals who are 55-to-75-years-of-age, who smoked the equivalent of one pack of cigarettes each day for 30 years and who have not quit smoking with 15 years. While LDCT has a sensitivity of 93.8%, its specificity has been shown to be 73.4%, according to the National Lung Cancer Screening Trial (LCST), the largest trial of lung cancer screening to date. The LCST showed a false positive rate of 3.8% for LDCT in the high-risk population it studied, leading to many unnecessary, often invasive and potentially harmful follow-up procedures in patients who test positive by LDCT but who do not have lung cancer. There is thus a pressing need to improve the specificity of the LDCT and thereby lowering its false positive rate. One approach toward addressing this need is the development of additional assays with a high specificity for lung cancer that can be used as an adjunct to LDCT. The highly fluorescent tetra (4-carboxyphenyl) porphyrin (TCPP) selectively binds to cancer cells compared to normal cells, and is thus uniquely suited for the development of a diagnostic label that can distinguish cancer cells from surrounding background cells. The standard of care for screening individuals at high risk for lung cancer consists of annual imaging of the chest using LDCT (1). Although extremely sensitive, LDCT has a high false positive rate leading to multiple reflex diagnostic procedures with associated risks for patients who ultimately test negative for cancer. Risks include additional high-dose radiation exposure, and complications and morbidity from invasive procedures such as thoracentesis, bronchoscopy, and core needle biopsy. The risk of adverse events and the added financial burden associated with these procedures is significant, resulting in a clear medical need for safer and less invasive reflex testing after positive LDCT results (2). Alternative testing methods would ideally complement the high sensitivity of LDCT by increasing the specificity, lowering the false positive rate and improving the positive predictive value of screening with a reasonably priced adjunct test.

Minimally invasive techniques in the form of liquid biopsies have been proposed for reflex lung cancer testing following positive LDCT results. Using a liquid biopsy, circulating tumor cells (CTCs) and free tumor nucleic acids can be collected from the patient's peripheral blood sample. The CTCs and nucleic acids are tested using molecular techniques such as next-generation sequencing (NGS) for the presence of cancer-associated gene mutations that could predict the presence of cancer and how the patient's tumor might respond to a specific targeted therapy (3). While these technologies can identify mutations in an estimated 50-75% of lung cancers (4,5), LDCT-positive patients whose tumors lack such specific gene abnormalities will have negative results from a liquid biopsy. In addition, CTCs are rare (as low as 1 cell per 10⁹ normal cells) and tumor nucleic acid concentrations are often below the limit of detection of most clinically available molecular testing methods (6). Thus, liquid biopsies have the potential to provide valuable treatment information about a patient's tumor genome but are better utilized at a later stage in the lung cancer diagnostic algorithm than tests aimed at early cancer diagnosis.

Liquid cytology testing of bronchial washings provides a sampling of potentially malignant cells for pathology review using the conventional sputum smear. The bronchoscopy procedures used to retrieve cells from a patient's airway are less invasive than a core needle lung tissue biopsy. However, there is still risk for adverse events such as hemorrhage (7). In addition, associated health care costs, particularly if performed on an inpatient basis, can be significant. Given that only a small minority (i.e., less than 4%) of LDCT-positive patients will be found to actually have lung cancer, there remains a medical need for alternative, economical, more accessible sources of malignant cells from the lung to provide diagnostic material.

Pathologists have performed routine cytological examination of sputum for decades as a non-invasive, rapid and specific detection method for lung cancer. In conventional sputum cytology, samples are stained and screened microscopically for malignant cells. However, conventional sputum cytology suffers from low (˜65%) sensitivity (8). Various methods to enhance sensitivity of sputum analysis have been attempted, including KRAS mutation testing. While KRAS testing can be both sensitive and specific if a patient's tumor is in fact KRAS mutated, only 15-20% of lung cancers actually harbor KRAS gene mutations. Thus, KRAS mutation-negative tumor cells will not be detected by this technique (9). An alternative DNA-based approach, referred to as automated sputum cytometry, utilizes special staining and computer-assisted image analysis to assess nuclear DNA characteristics of sputum epithelial cells for malignancy-associated changes. While this technique is somewhat more sensitive than conventional cytology, its specificity is only ˜50% (10).

### BRIEF SUMMARY OF THE INVENTION

One embodiment of the present invention provides for a method of predicting the likelihood of lung disease in a subject, the method comprising the steps of labeling an ex-vivo sputum sample with one or more of the following i) a first labeled probe that binds a biomarker expressed on a white blood cell population of sputum cells; ii) a second labeled probe is selected from the group consisting of: a granulocyte probe that binds a biomarker expressed on a granulocyte cell population of sputum cells, a T-cell probe that binds a biomarker expressed on a T-cell cell population of sputum cells, a B-cell probe that binds a biomarker expressed on a B-cell cell population of sputum cells, or any combination thereof; iii) a third labeled probe that binds a biomarker on a macrophage cell population; iv) a fourth labeled probe that binds to a disease related cell in the sputum sample; v) a fifth labeled probe that binds to a biomarker expressed on an epithelial cell population of sputum cells; and vi) a sixth labeled probe that binds to a cell surface biomarker expressed on an epithelial cell population of sputum cells. The labelled sputum sample is analyzed, for example, flow cytometrically analyzed to obtain data comprising per cell cytometric data based upon a mean fluorescent signature of any of the i)-vi) labeled probes. The per cell data is detected to determine the likelihood of lung disease in a subject based upon a profile of a presence or absence of labeled probes in the per cell labelled data. The data obtained can be further analyzed to identify the presence or absence of a biomarker in a sputum sample. For example, the disease related cells may be lung cancer cells or tumor associated immune cells. The lung disease may be one selected from the group consisting of asthma, COPD, influenza, chronic bronchitis, tuberculosis, cystic fibrosis, pneumonia, graft vs. hose disease and lung cancer. Further, the sputum cells that are labelled may be fixed or non-fixed.

The data collected from the labelled sputum sample can be characterized by the populations of cells and biomarkers therefrom identified. For example, a ratio of the sputum cells in the data collected from the labelled sputum sample is determined that are negative for i) as compared to the sputum cells that are positive for i) to identify a biomarker 1. In one example, a ratio of less than 2 indicates the sputum sample is positive for biomarker 1. In one embodiment, the positive biomarker 1 has a sensitivity of at least about 80% and a specificity of at least 50% to distinguish a lung cancer (c) sputum sample from a high risk (HR) sputum sample with the application of biomarker 1. Wherein the sensitivity is at least: 85%, 90% or 95% and the specificity is at least 55%, 60%, 65%, 70%, 75%, 80%, 85%, 90% or 95%.

In another example, from the data collected from the labeled sputum sample, identifying the sputum cells that are negative for i) and positive for iv) and v) to identify a biomarker 2. For example, a percentage of sputum cells negative for i) and positive for iv) and v) that is greater than 0.03% indicates the sputum sample is positive for biomarker 2. In one embodiment, the positive biomarker 2 has a sensitivity of at least 90% and a specificity of at least 50% to distinguish a lung cancer (c) sputum sample from a high risk (HR) sputum sample with the application of biomarker 2. Wherein the sensitivity is at least: 80%, 85% or 95% and the specificity is at least 55%, 60%, 65%, 70%, 75%, 80%, 85%, 90% or 95%.

In another example, a biomarker 3 is identified when the sputum cells are positive for i), iii) and display FITC autofluorescence. For example, a percentage of sputum cells positive for i), iii) and display FITC autofluorescence that is greater than 0.03% indicates the sputum sample is positive for biomarker 3. In one embodiment the positive biomarker 3 has a sensitivity of at least 60% and a specificity of at least 70% to distinguish a lung cancer (c) sputum sample from a high risk (HR) sputum sample with the application of biomarker 3. Wherein the sensitivity is at least: 65%, 70%, 75%. 80%, 85%, 90% or 95% and the specificity is at least 65%, 70%, 75%, 80%, 85%, 90% or 95%.

In another example, a biomarker 4 is identified when the sputum cells are negative for i) and positive for v) and vi) to identify a biomarker 4. For example, the percentage of cells negative for i) and positive for v) and vi) of more than 2% indicates the sample is positive for biomarker 4. In one embodiment, the positive biomarker 4 has a sensitivity of at least 70% and a specificity of at least 70% to distinguish a lung cancer (c) sputum sample from a high risk (HR) sputum sample with the application of biomarker 3. Wherein the sensitivity is at least: 80%, 85%, 90% or 95% and the specificity is at least 65%, 70%, 75%, 80%, 85%, 90% or 95%.

In another embodiment more than one biomarker can be combined such as a combination of the positive biomarker 1 and the positive biomarker 2 to produce have a sensitivity of at least 80% and a specificity of at least 80% to distinguish a lung cancer (c) sputum sample from a high risk (HR) sputum sample with the application of biomarker 1 and 2. Further, the combination of positive biomarkers 1, 2, and 3 to produce a sensitivity of at least 80% and a specificity of at least 80% to distinguish a lung cancer (c) sputum sample from a high risk (HR) sputum sample with the application of biomarkers 1-3. Further still, the positive biomarkers 1-4 produce a sensitivity of at least 70% and a specificity of at least 75% to distinguish a lung cancer (c) sputum sample from a high risk (HR) sputum sample with the application of biomarkers 1-4. Wherein the sensitivity is at least: 70%, 75%, 80%, 85%, 90% or 95% and the specificity is at least 65%, 70%, 75%, 80%, 85%, 90% or 95%.

In one embodiment, the flow cytometric analysis may include one or more of the following: excluding from data analysis those cells that have a diameter of less than about 5 μm and greater than about 30 μm, those cells that are dead cells and cell clumps of more than one.

In another embodiment, the first labeled probe that binds a biomarker expressed on a white blood cell population of sputum cells may be a CD45 antibody or fragment thereof.

In another embodiment, the second labeled probe is one or more of the following added either individually or in combination to the sputum sample: the granulocyte probe that binds a biomarker expressed on a granulocyte cell population of sputum cells and may be selected from a CD66b antibody or fragment thereof, the T-cell probe that binds a biomarker expressed on a T-cell cell population of sputum cells is a CD3 antibody or fragment thereof, the B-cell probe that binds a biomarker expressed on a B-cell cell population of sputum cells is a CD19 antibody or fragment thereof.

In another embodiment, the third labeled probe that binds a biomarker on a macrophage cell population of sputum cells is a CD206 antibody or fragment thereof.

In yet another embodiment, the fourth labeled probe that binds to a disease related cell in the sputum sample is a tetra (4-carboxyphenyl) porphyrin (TCPP).

In yet another embodiment, the fifth labeled probe that binds to a biomarker expressed on an epithelial cell population of sputum cells is a panCytokeratin antibody or fragment thereof.

In a further embodiment, the sixth labeled probe that binds to a cell surface biomarker expressed on an epithelial cell population of sputum cells is an EpCam antibody or fragment thereof.

The data collected may comprise per cell cytometric data based upon a mean fluorescent signature of any of the i)-vi) labeled probes to produce a sputum sample signature. The sputum sample signature identifies the health of the lung and/or lung disease. The lung disease may be selected from the group consisting of asthma, COPD, influenza, chronic bronchitis, tuberculosis, cystic fibrosis, pneumonia, graft vs. hose disease and lung cancer. Further still, the sputum sample signature is compared to a database of control sputum sample signatures (non-diseased) and lung disease sample signatures to identify lung disease. In some embodiments of the present invention, results are classified using a trained algorithm. Trained algorithms of the present invention include algorithms that have been developed using a reference set of known sputum samples from subject at high risk of developing the disease, sputum samples for subjects confirmed to have the disease and sputum samples from subjects identified as normal (not having the disease or at high risk of developing the disease). Algorithms suitable for categorization of samples include but are not limited to k-nearest neighbor algorithms, concept vector algorithms, naive bayesian algorithms, neural network algorithms, hidden markov model algorithms, genetic algorithms, and mutual information feature selection algorithms or any combination thereof. In some cases, trained algorithms of an embodiment of the present invention may incorporate data other than sputum sample signatures or per cell cytometric data or mean fluorescent signature such as diagnosis by cytologists or pathologists or information about the medical history of the subject. In a programmed computer, the data is input to a trained algorithm to generate a classification of the sputum sample as high probability, intermediate probability or low probability of having the lung disease and electronically outputting a report that identifies said classification of said sputum sample for the lung disease.

One embodiment of the present invention provides for a first reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome; and a fluorochrome-conjugated antibodies directed against cell's markers selected from; ii) EpCAM, and/or panCytokeratin, and iii) CD45, CD206, CD3, CD19, CD66b or any combination thereof.

Another embodiment of the present invention provides for a second reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome and fluorochrome-conjugated antibodies directed against the following cell's markers; ii) EpCAM and/or panCytokeratin, and iii) CD45.

Another embodiment of the present invention provides for a third reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome; and fluorochrome-conjugated antibodies directed against one or more of the following cell's markers; CD45, CD206, CD3, CD19, and CD66b.

Yet another embodiment provides for a method of predicting the likelihood of lung disease in a subject, comprising the steps of labeling an ex-vivo sputum sample with i) a labeled probe that binds to a disease related cell in the sputum sample and ii) one or more fluorochrome-conjugated probes directed against a sputum cell's markers. The labelled sputum sample is flow cytometrically analyzed to obtain data comprising per cell cytometric data based upon a mean fluorescent signature of any of the i)-ii) labeled probes. From the per cell data detecting the likelihood of lung disease in a subject based upon a profile of a presence or absence of i) and ii) in the per cell labelled data. The data comprising per cell cytometric data can be based upon a mean fluorescent signature of any of the i)-ii) produces a sputum sample signature. In one embodiment, the sputum sample signature identifies the lung disease for example, the lung disease is selected from the group consisting of asthma, COPD, influenza, chronic bronchitis, tuberculosis, cystic fibrosis, pneumonia, graft vs. hose disease and lung cancer. Further still, the sputum sample signature is compared to a database of control sputum sample signatures (non-diseased) and lung disease sample signatures to identify the lung disease from the labelled sputum sample. In one embodiment, the labeled probe that binds to the disease related cell in the sputum sample is a tetra (4-carboxyphenyl) porphyrin (TCPP).

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 A-B illustrate cytospins from dissociated sputum cells. Wright-Giemsa-stained cytospin slides of processed sputum cells before staining with antibodies or TCPP.

FIG. 1C-E illustrate a flow cytometry-based system having a light source and detector for analyzing optical properties from a cell or particle with the forward scatter (FSC) and side scatter (SSC) being identified as exemplary optical properties for a cell or particle passing through the zone of the laser light source over time with the measurement of a pulse height and area as measurements in the histogram shown in FIG. 1D.

FIG. 2A-I illustrate flow cytometry dot plots FIG. 2 (A-F) and contour plots FIG. 2 (G-I) of beads (FIG. 2A and FIG. 2G) and cells (FIG. 2 B-F, FIG. 2H, and FIG. 2I).

FIG. 3A-K illustrate dot plots and contour plots for the identification and characterization of hematopoietic cells in sputum.

FIG. 4 A-G illustrate dot plots (FIG. 4A, FIG. 4C, FIG. 4F-G) and histograms (FIG. 4B, FIG. 4D and FIG. 4E) of CD45^{positive} sputum cells exposed to either CD66b probe or CD206 probe.

FIG. 5 is a graph illustrating the number of macrophages/slide on the y(axis) shown as solid circle with “x” inside and CD45^{positive}/CD206^{positive} cells shown as solid circle and sample number on the x(axis) that the presence of a CD206^{positive} cell population coincides with the presence of numerous macrophages on a sputum smear.

FIG. 6 illustrates a flow chart of sputum sample preparation for analysis. HCC15 cancer cells were labeled with CellMask™ Green (step 1) while, in a different tube, dissociated sputum cells were stained with a PE-labeled anti-CD45 antibody (step 2).

FIG. 7A-F illustrate dot plots of sputum cells with FIG. 7A representing the CD45 gate, FIG. 7B representing a TCPP gate in CD45^{positive} cells and FIG. 7C representing the TCPP gate in the CD45^{negative} cells and FIG. 7D-F representing the isotype control treated unstained sputum cells and stained sputum cells.

FIG. 8A-B illustrate a preliminary, comparative analysis of sputum samples obtained from healthy volunteers and high-risk patients with and without lung cancer. Five sputum samples from different donors were analyzed similar to the experiment detailed in FIG. 6 and FIG. 7. The open dots represent a sample from a healthy volunteer (H), the black dots represent a sample from high-risk patient without cancer (HR) and the dot with x represents a sample from a confirmed lung cancer patient (C). FIG. 8A illustrates the total numbers of CD45^{negative} (left) and CD45^{positive} cells (right) within each sample analyzed. FIG. 8B illustrates the proportion of TCPP^{positive} cells within the CD45^{negative} (left) and CD45^{positive} cells (right) within each sample analyzed.

FIG. 9A-F illustrate dot plots for one strategy for analyzing sputum cells for the presence of TCPP^{positive} cells according to one embodiment of the present invention.

FIG. 10A-B illustrate QC bead and sputum sample tube #6 as described in the protocol are analyzed via flow cytometry and the resulting dot plots. FIG. 10A illustrates bead size exclusion (“BSE”) gate (box) which is first set on the profile obtained from running QC beads. FIG. 10B illustrates the BSE gate applied to all sputum samples.

FIG. 11A-F illustrate sputum samples that are analyzed via flow cytometry and the resulting dot plots for determination of sputum cells unstained (tube #4) as illustrated in FIG. 11A, FIG. 11B and stained sputum cells (tube #6) as in FIG. 11C to identify live cells (LC) as illustrated in the box of FIG. 11C and single cells (SC) as illustrated in FIG. 11D. FIG. 11E and FIG. 11F illustrate dot plots of sputum cells to set the isotype control FIG. 11E and the CD45^{positive} and CD45^{negative} populations of cells remaining after application of the BSE, LC, SC gates.

FIG. 12A-C illustrate CD45^{positive} cell analysis of a sputum sample of tube #6. All profiles depict CD45^{positive} cells that have been selected through the BSE, LC and SC gates.

FIG. 13A-B illustrate dot plot of isotype control for FITC/Alexa488 (F/A) (tube #5) and cells treated with probe for CD66b/CD3/CD19 cell marker conjugated with (F/A) (tube #6).

FIG. 14A-B illustrate dot plot of PE-CF594 isotype control (tube #5) and cells treated with probe for CD206 cell marker conjugated with PE-CF594.

FIG. 15A-B illustrate a dot plot of the isotype control for FITC/Alexa488 on the y axis and PE-CF594 on the x(axis) of sputum cells (tube #5). A double-negative gate or population 1 parameter is established. Presented are a dot plot FIG. 15A and a pseudocolor plot FIG. 15B from the isotype control, that have been gated through the BSE, LC and CD45^{positive} cell gates. The horizontal dotted line represents the FITC/Alexa488 positive/negative cut off determined in FIG. 13, whereas the vertical dotted line is derived from the PE-CF594 positive/negative cut off determined in FIG. 14.

FIG. 16A-B illustrate dot plot (A) and a pseudocolor plot (B) from a sputum sample as per tube #6 and measured for the mean fluorescence intensity from a cocktail (CD66b/CD3/CD19-FITC/Alexa488 antibodies (y-axis) and marker CD206 conjugated with PE-CF594 (x-axis). CD45^{positive} cells are shown that were also selected through the BSE, LC and SC gates. The same population 1 (solid interior box) and the cut offs (dotted lines), as drawn in FIG. 15 are applied to these profiles.

FIG. 17A-C illustrate pseudocolor plots generated from the sputum CD45^{positive} tube from two samples (A and B are the same) and the gates set for populations 2-6 of the sputum sample of FIG. 16 are applied. All plots show CD45^{positive} sputum cells that have been gated through the BSE, LC and SC gates. The horizontal and vertical dotted lines were set on the isotype controls (not shown). FIG. 17A-B demonstrate in a drawing of gates 4 and 5, when the FITC mean fluorescence intensity of population 5 is intermediate and crossing the horizontal cut-off line. FIG. 17C illustrates a population 6 upper-right box.

FIG. 18 illustrates a graph of percent (%) of all blood (CD45^{positive}) cells in a sputum sample on the y axis and profile type 1, 2, and 3 on the x axis. The signature illustrated is for Profile 1 for CD45^{positive} cells for high risk (HR) samples.

FIG. 19A-C illustrate graphs for signatures 1-3 for CD45^{positive} sputum cells from HR and cancer cells and analysis of population 6 as a percent of all CD45^{positive} blood cells for HR and C sputum sample.

FIG. 20A-D illustrate dot plots of CD45^{negative} sputum samples with gates drawn for the different epithelial subpopulations in sputum.

FIG. 21A-B illustrate a dot plot of isotype control for FITC/Alexa488 and CD45^{negative} sputum cells (tube #5) and sputum cells labeled with panCytokeratin/Alexas488 (tube #7). The cut off for positive FITC/Alexa488 staining in CD45⁻ sputum cells is determined.

FIG. 22A-B illustrate dot plot of isotype control for PE-CF594 and sputum cells (tube #5) and sputum cells labeled with EpCAM-PE-CF594 (tube #7). Determining the cut off for positive PE-CF594 staining in CD45^{negative} sputum cells and sputum.

FIG. 23A-B illustrate dot plots of CD45^{negative} cells with isotype controls (tube #5), that have been gated through the BSE, LC and CD45 cell gates. The horizontal dotted line represents the FITC/Alexa488 positive/negative cut off determined in FIG. 21, whereas the vertical dotted line is derived from the PE-CF594 positive/negative cut off determined in FIG. 22.

FIG. 24A-B illustrate dot plots of sputum cells and gates for populations 2-9 of the CD45^{negative} cells.

FIG. 25 illustrates a separate graph of CD45^{negative} dot plots for profile 1-4 with different signatures for populations 1-9.

FIG. 26 illustrates a signature for profile 1 across the median of population 1, population 2, population 5 and panCK++.

FIG. 27 illustrates a comparison of signature 1-4 for CD45^{negative} cells from a sputum sample from subjects classified as at high risk for developing lung cancer and sputum samples from subjects classified as having lung cancer.

FIG. 28A-B illustrate a sensitivity of 80% and a specificity of 85% for application of the biomarker resulting from the amount of PanCK++(populations 3+4+9) as a percentage (%) of all CD45^{negative} cells from a sputum sample.

FIG. 29A-C illustrate cancer risk analysis of cells in a sputum sample from HR and C sputum samples to determine the ratio of CD45^{negative}/CD45^{Positive} (biomarker 1) of the cells in the sputum sample.

FIG. 30A-B illustrate specificity of 90% and sensitivity of 54% for the identification of samples as from a lung cancer patient or a subject at high risk of developing lung cancer with the application of biomarker 1 to the sputum sample analyzed.

FIG. 31A-C illustrate cancer risk analysis of CD45^{negative} cells in a sputum sample (tube #7) positively labeled with TCPP (biomarker 2).

FIG. 32A-B illustrate specificity of 63% and sensitivity of 100% for the identification of samples as from a lung cancer patient or a subject at high risk of developing lung cancer with the application of Biomarker 2 to the sputum sample analyzed.

FIG. 33A-C illustrate a combination of biomarker 1 and biomarker 2 as identified in FIG. 25 and FIG. 27 to analyze a sputum sample for HR and C sputum samples to yield a sensitivity of 90% and a specificity of 90% for the according to one embodiment of the present invention for the identification of samples as from a lung cancer patient or a subject at high risk of developing lung cancer with the application of biomarker 1+2 to the sputum sample analyzed.

FIG. 34A-C illustrate dot plots from CD45^{positive} cells to identify amount of cells in population 6 (biomarker 3) from HR and C sputum samples as a % of all CD45+ cells in the sample.

FIG. 35A-B illustrate specificity of 88% and sensitivity of 60% for the identification of samples as from a lung cancer patient or a subject at high risk of developing lung cancer with the application of biomarker 3 to the sputum sample analyzed.

FIG. 36A-B illustrate cancer risk analysis of CD45^{negative} cells from a sputum sample that are also panCytokeratin^{positive} (biomarker 4) found in populations 3+4 and 9 from HR and C sputum samples.

FIG. 37A-B illustrate specificity of 83% and sensitivity of 80% for the identification of samples as from a lung cancer patient or a subject at high risk of developing lung cancer with the application of biomarker 4 to the sputum sample analyzed.

FIG. 38A-E illustrate cancer risk analysis of cells from a sputum sample with the application of biomarkers 1-4 to HR and C sputum samples with specificity of 98% and sensitivity of 78%.

FIG. 39 illustrate a screening flow chart for lung health of subjects that include a system and method for fractionating cell populations from the lung as described herein and an algorithm for the classification of the sputum sample as high risk, intermediate risk and low risk for lung disease.

### DETAILED DESCRIPTION OF THE INVENTION

Furthermore, the following terms shall have the definitions set out below. It is understood that in the event a specific term is not defined herein below, that term shall have a meaning within its typical use within context by those of ordinary skill in the art.

It is to be noted that as used herein and in the appended claims, the singular forms “a,” “and” and “the” include plural references unless the context clearly dictates otherwise.

The term “calibrate” means setting the sensitivity of the machine against the control reagents.

The term “compensation” means samples are compared against controls to determine background.

The term “fractionate” or “fractionated” means selecting a subset of events to further analyze. One example of fractionating is with “gates” to exclude/include data during analysis.

The term “gate” means boundaries are placed around populations of cells with common characteristics, usually forward scatter, side scatter, and marker expression, to investigate and to quantify these populations of interest.

The term “probe” means a ligand, peptide, antibody or fragment thereof that has affinity for and binds to a biomarker on the surface of a cell or particle or to a marker within the cell or particle.

Porphyrins concentrate in all types of cancer cells. In addition, certain porphyrins are naturally fluorescent, with a characteristic photon emission profile. A porphyrin composition is described herein for use in a high-throughput assay (especially a flow cytometric assay) to distinguish fluorescence of porphyrins that label cancer cells or cells associated with a disease state from surrounding background cells (11).

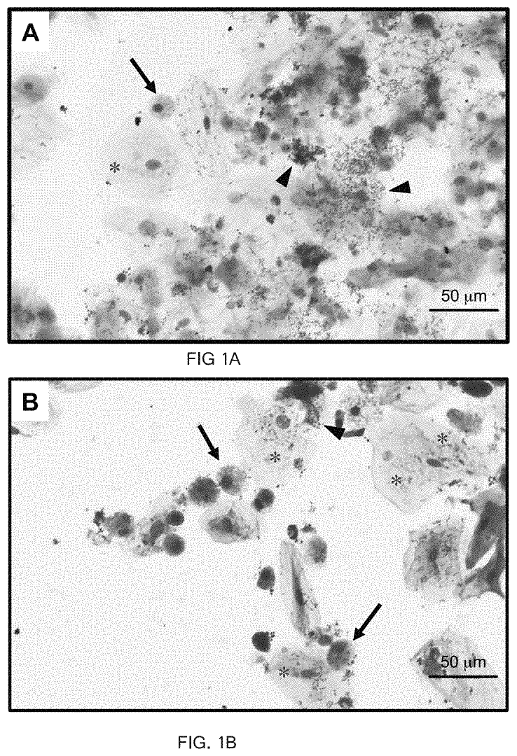

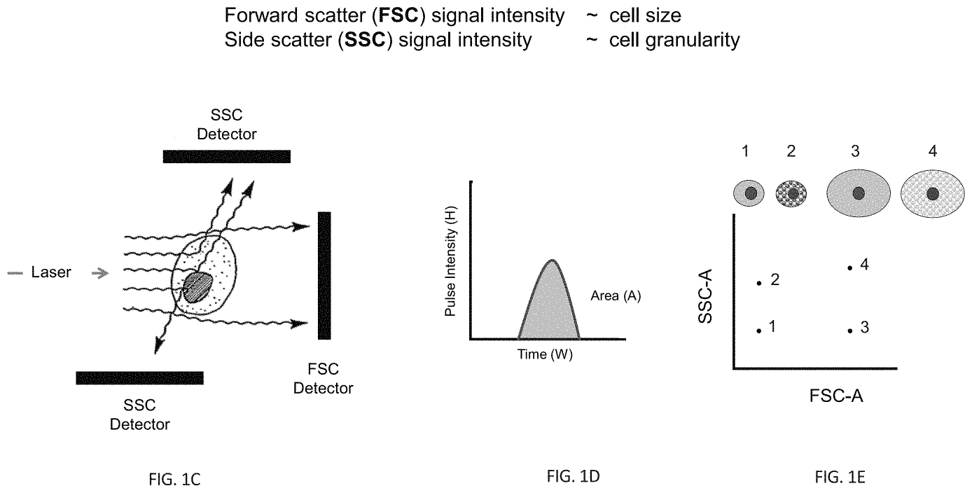

Referring now to FIG. 1 A-B, cytospins from dissociated sputum cells are illustrated. Wright-Giemsa-stained cytospin slides of processed sputum cells before staining with antibodies or TCPP are illustrated in FIG. 1A. FIG. 1A contains too many buccal epithelial cells (BEC)s (some of which are indicated by a * symbol). Macrophages are indicated by an arrow and debris by an arrow. FIG. 1B shows the presence of less debris (indicated by arrowheads) allowing easier identification of BECs and macrophages on the slide.

In flow cytometry each cell or particle is hydrodynamically focused to a photocell. Each cell or particle passes through one or more beams of light as the cell/particle passes through the photocell. Light scattering or fluorescence (FL) emission (if the cell or particle is labeled with a fluorophore) provides information about the cell's/particle's properties. Lasers are the most commonly used light sources in modern flow cytometry. Lasers produce a single wavelength of light (a laser line) at discrete frequencies (coherent light). They are available at different wavelengths ranging from ultraviolet to far red and have a variable range of power levels (photon output/time). Light that is scattered in the forward direction, typically up to 20° offset from the laser beam's axis, is collected by a photomultiplier tube (PMT) or photodiode known as the forward-scatter (FSC) channel. The FSC equates roughly to the cell's/particle's size. Typically, larger cells refract more light than smaller cells. Light measured at an approximately 90° angle to the excitation line is called side scatter (SSC). The SSC channel provides information about the relative complexity (for example, granularity and internal structures) of a cell or particle. Both FSC and SSC are unique for every cell or particle, and a combination of the two may be used to roughly differentiate cell types in a heterogeneous sample such as blood, sputum, for example, but not limited thereto. An event is identified when a cell or particle passes through the laser beam and a signal is generated as a function of time. For FSC and SSC, the time that the cell or particle spends in the laser is measured as the width “W” of the event while the maximum height of the current output measured by the photomultiplier tube is the height “H” and the area “A” represents the integral of the pulse generated by the cell or particle passing the interrogation point of a laser beam in the cytometer. As used herein cell and particle may each be recorded as an event when passing through the beam of light in the photocell.

Referring now to FIG. 1C a light-scatter profile (where the forward side scatter (FSC) represents cell size and side scatter (SSC) represents granularity) where “A” represents the integral of the pulse generated by the cell or particle passing the interrogation point of a cytometer is illustrated. FIG. 1D is a resulting histogram of laser pulse intensity (H) on the y(axis) and Time (W) on the x(axis) with the area under the curve indicated as (A). FIG. 1E illustrates a SSC-A vs. FSC-A plot of cells having different granularity and size on the plot. A light-scatter profile (where the forward side scatter (FSC) represents cell size and side scatter (SSC) represents granularity) where “A” represents the integral of the pulse generated by the cell or particle passing the interrogation point of a cytometer.

### Light-Scatter Gates to Enrich for RFCs.

Specialized airway epithelium cells and glandular cells lining the bronchi secrete mucus. The mucus produced deep within the lung can contain a large variety of cells that are recycled from the lung tissue, including epithelial cells, alveolar cells, macrophages and other hematopoietic (blood) cells (17). The mucus also contains non-cellular material, which is especially noticeable in lungs from people who smoke, live in highly polluted areas or are exposed to other airway allergens (such as pollens). When mucus originating from within the lung is coughed up, it is called sputum. Sputum is often mixed with saliva produced in the oral cavity that contains many BECs (or cheek cells), which adds another cellular component to an already complex tissue sample (see FIG. 1).

As opposed to microscopy, flow cytometry can provide for multidimensional information and/or more exacting information regarding cell populations from sputum, because it allows the elimination of debris and cells that are not of interest based on size, granularity and/or fluorescence markers, thereby enriching the sample for cells of interest. To enrich for red fluorescent cells (RFC)s in sputum cell analysis, the first step is to approximate the size (diameter) of RFCs; anything smaller or larger is excluded. RFCs are the cells with the highest TCPP uptake, i.e., cancer cells and cancer-associated macrophages, because both cell types take up more TCPP than any other cell type (18-22). The size of lung cancer cells may vary and depend on the type of cancer but is not likely to significantly differ from cultured lung cancer cells. A literature search (Table 1) reveals that the diameter of HCC15 lung cancer cells is 20-30 μm, for example, while the diameter of alveolar macrophages is measured to be 21 μm. Of special interest are the macrophages and lymphocytes, since specific subpopulations of each of these cell types are known to alter their function when associated with cancers (23-26). However, RBC (6-8 μm) and anything smaller (debris), as well as BECs (65 μm) and anything larger can be excluded from further analysis.

TABLE 1

| Cell Type | Diameter (μm) | Reference |
|---|---|---|
| Hematopoietic cells | | |
| Erythrocytes | 6-8 | Wheater et al. (43) |
| Granulocytes | 9-12 | Wheater et al. (43) |
| Monocytes | 14-17 | Wheater et al. (43) |
| Lymphocytes | 7-8 | Wheater et al. (43) |
| Other | | |
| Alveolar macrophages | 21 | Krombach et al. (44) |
| Type I alveolar epithelial | up to 50 | Kini (45) |
| cell (lung cells) | | |
| Type II alveolar epithelial | 9-15 | Kini (45) |
| cells (lung cells) | | |
| Buccal epithelial cells | 65 | Paszkiewicz et al. (14) |
| (cheek cells) | | |
| HCC15 lung cancer cells | 20-30 | Fillmore et al. (46) |

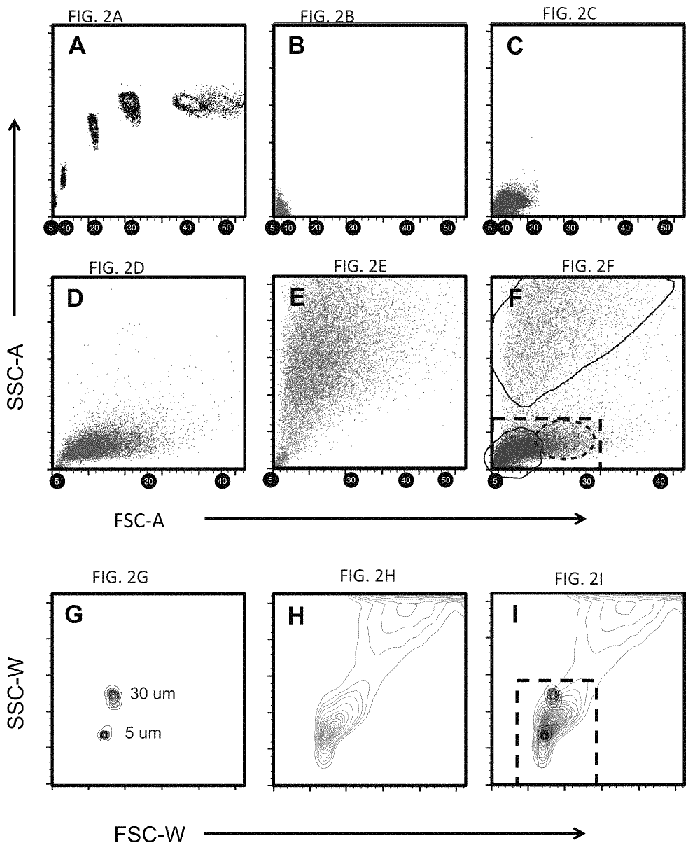

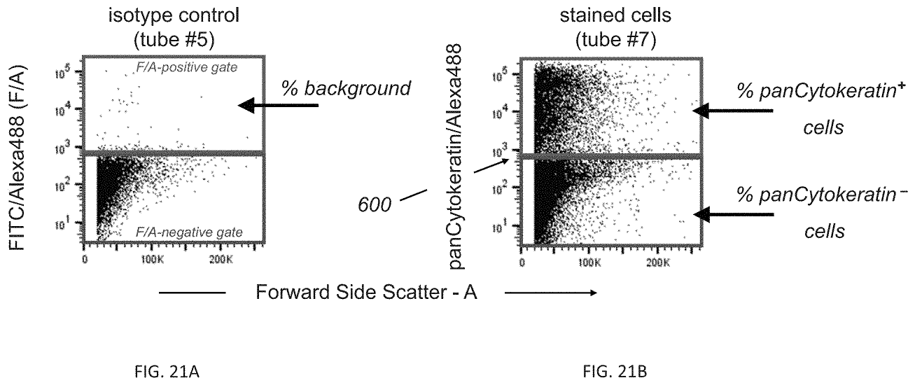

Referring now to FIG. 2 A-I, flow cytometric profiles illustrating cells having SSC and FSC signatures are shown. Depicted are flow cytometry dot plots FIG. 2 A-F and contour plots FIG. 2 G-I of beads (FIG. 2A and FIG. 2G) and cells (FIG. 2 B-F, FIG. 2H, and FIG. 2I). FIG. 2A is a light-scatter plot showing from left to right 5, 10, 20, 30 and 50 μm beads. The size of the individual beads is manually drawn onto the horizontal FSC axis and carried over to figures FIG. 2 B-F. The SSC was kept initially low, so that cells with a higher SSC than expected could be visualized. FIG. 2B is a light-scatter plot of red blood cells (RBC)s, stained with CellMask™ Orange. FIG. 2C is a light-scatter plot of white blood cells (WBC)s stained with CellMask™ Far Red. FIG. 2D is a light-scatter plot of squamous cell lung carcinoma cells (HCC15) cells stained CellMask™ Orange. FIG. 2E is a light-scatter plot of buccal epithelial cells (BEC)s stained with CellMask™ Green. FIG. 2F is a light-scatter profile of WBCs (positioned as in FIG. 2C), HCC15 cells (positioned as in FIG. 2D) and BECs (positioned as in FIG. 2E) put together in one tube for analysis. The striped box in FIG. 2F indicates the light-scatter gate that includes the cells of interest; they include everything of 5 to 30 μm in size. FIG. 2G depicts 5 μm (lower) and 30 μm (upper) beads in an FSC-Wx SSC-W light-scatter contour plot. FIG. 2H is a FSC-Wx SSC-W light-scatter contour plot of BECs stained with CellMask™ Green (as in FIG. 2E). FIG. 21 illustrates the combined cell populations (WBCs, BECs and HCC15 displayed in an FSC-Wx SSC-W light-scatter contour plot. The separation between the BECs (cells larger than 30 μm and located outside of the broken line box) and cells of interest (cells smaller than 30 μm located within the broken line box) is clearly visible. The broken line box indicates the Wx W gate and identifies the population of interest that allows for easy exclusion of most BECs.

In one embodiment, debris and BECs are excluded from a population of cells to be further analyzed. Standard-size beads (5, 10, 20 and 50 μm) are used in a light-scatter profile (where the forward side scatter (FSC) represents cell size and side scatter (SSC) represents granularity; FIG. 2A). To confirm that these beads would indeed predict cell sizes in accordance with the information presented in Table 1, the beads are compared to RBCs, WBCs and BECs isolated from healthy volunteers, as well as cultured HCC15 lung cancer cells. The different cell types are labeled with CellMask™ dyes of different colors, so that they can be analyzed separately (FIG. 2 B-E) and in combination (FIG. 2F). As illustrated in FIG. 2B and predicted from the literature (Table 1), RBCs coincide with the smallest beads. Similarly, WBCs range from approximately 10 to 20 μm in size (FIG. 2C) while the majority of HCC15 cells are smaller than 30 μm in diameter (FIG. 2D). When saliva was analyzed through the flow cytometer (which consists mostly of BECs), contrary to expectations, as informed by the literature, the majority of BECs are projected as cells of 30 μm or less (FIG. 2E) and not as cells larger than 50 μm. These results demonstrate that size can be used to exclude debris (by eliminating everything that is smaller or equal in size to the 5 μm beads), but size cannot be used to exclude BECs.

BECs demonstrate very high SSC characteristics that made them distinct from WBCs and HCC15 cells (FIG. 2F). The SSC and FSC are translated by the flow cytometer as electronic signals with height (H), width (W) and an area under the curve (A) values. By looking at the various combinations of SSC and FSC parameters, the SSC-W and FSC-W resulted in a profile that allowed elimination of most BECs by setting a gate around the cells that showed a lower SSC-W than the 30 μm beads (FIG. 2 G-I).

Sub-Fractionation of Hematopoietic Cells into Discrete Populations

Another aspect of sputum analysis by flow cytometry is the characterization of the various hematopoietic (blood) cell populations. The common WBC marker CD45 is expressed on the cell surface of all WBCs. Using a probe, for example an antibody, directed against the CD45 antigen, hematopoietic cells (CD45^{positive} cells) can be distinguished from other cells, including normal lung epithelial cells and potential lung cancer cells (CD45^{negative} cells). To identify the specific hematopoietic subpopulations in sputum we used additional probes, for example, antibodies directed at granulocytes (CD66b), macrophages (HLA-DR, CD11b, CD11c, CD206) and lymphocytes (CD3 and CD19). Table 2 identifies exemplary probes and fluorophores.

TABLE 2

| Marker | Antibody/Dye | Fluorophore | | Laser source: | Tube #1 | Tube #2 |
|---|---|---|---|---|---|---|
| Dead cells | Viability Stain | BV510 | | 405 nm | ✓ | ✓ |
| Cancer (associated) cells | TCPP | APC | | 633 nm | ✓ | ✓ |
| Leukocytes | CD45 | PE | | 561 nm | ✓ | ✓ |
| Granulocytes | CD66b | FITC | | | ✓ | |
| T Cells | CD3 | Alexa488 | {close oversize bracket} | 488 nm | | |
| B Cells | CD19 | Alexa488 | | | | |
| Macrophage | CD206 | PE-CF594 (Tx-Red) | | | ✓ | |
| | | | {close oversize bracket} | 561 nm | | |
| Epithelial cells | EpCAM | PE-CF594 (Tx-Red) | | | | ✓ |
| | Cytokeratin (CK) | FITC | | 488 nm | | ✓ |

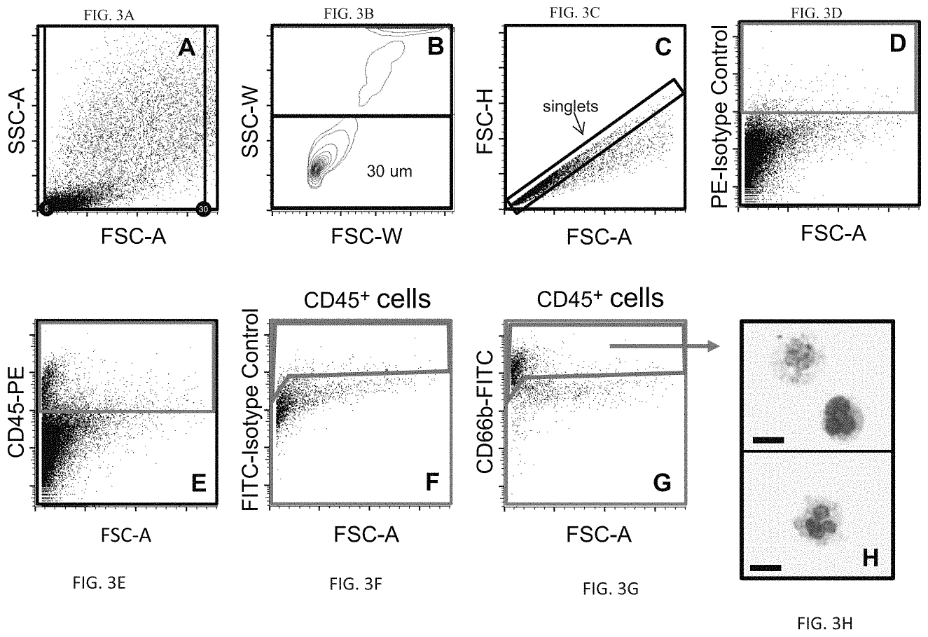

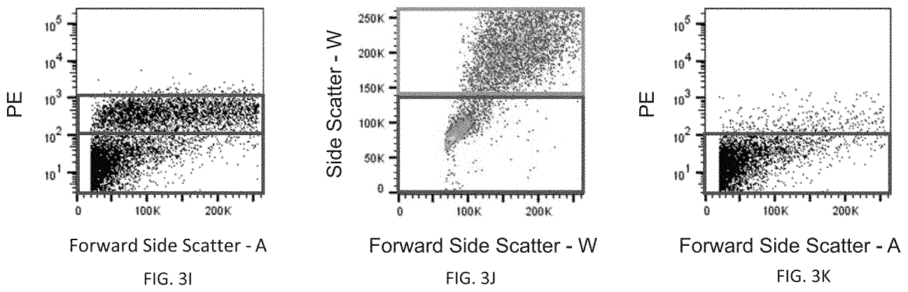

Referring now to FIG. 3A-K, identification and characterization of hematopoietic cells in sputum is illustrated. FIG. 3A illustrates sputum cells presented in a light-scatter plot of FCS-A v SSC-A. The black balls with the numbers on the x-axis represent the size of the beads used to set up this light-scatter gate that excludes debris and BECs, i.e., everything smaller than the 5 μm beads (vertical line to the left) and everything greater than 30 μm (vertical line to the right). FIG. 3B illustrates a FSC-Wx SSC-W contour plot of the cells within the light-scatter gate of FIG. 3A (where “W” represents the width of the signal). The size exclusion gate of 30 μm is identified as the horizontal line such that any cell detected in the upper box is larger than 30 μm. FIG. 3C depicts a FSC-A v FSC-H dot plot with the cells selected by the W×W gate shown in FIG. 3B where “H” represents the maximum amount of current output by the photo multiplier tube that detects the light from the laser of the cytometer. The indicated gate rectangle includes all single cells, while cell doublets are excluded. FIG. 3D illustrates dot plot of sputum cells, previously selected from the light-scatter gates depicted in FIG. 3A-C, stained with the PE-isotype control to determine the gate for CD45-specificity (indicated by the upper box). FIG. 3E illustrates a dot plot of sputum cells, previously selected from the light-scatter gates depicted in FIG. 3A-C, wherein the cells are stained with an anti-CD45-PE antibody. All cells expressing the CD45 antigen (CD45^{positive} cells) are captured in the upper box. Cells in the CD45^{positive} upper box/gate were then further analyzed for expression of CD66b. The background fluorescence of the anti-CD66 antibody is shown in FIG. 3F based upon staining with a FITC-Isotype control. FIG. 3G indicates CD45^{positive} cells stained with anti-CD66b. The CD45^{positive}CD66b^{positive} cells are indicated by the upper box. FIG. 3H is Wright-Giemsa staining of cells sorted from the upper box in FIG. 3G. FIG. 3I illustrates dot plot showing unstained sputum cells, selected only through the BSE gate. This particular sample shows a large subpopulation of cells falling within the box that shows an intermediate staining in the PE channel, the channel used to detect CD45 expression. The presence of this subpopulation makes it difficult to determine where to set the cut off for separating the sample into CD45^{negative} and CD45^{positive} cells. FIG. 3J illustrates a dot plot showing a W×W gate of the same sample as in FIG. 3I. The cells in the lower box (the W×W gate) are the cells of interest, while the cells captured in the upper box are SECs, which need to be excluded to reveal the true unstained sputum population of interest. FIG. 3K illustrates unstained sputum cells selected through the BSE gate and the W×W gate: the negative population is clearly identifiable and the CD45^{negative} gate having a mean fluorescence intensity that falls below the horizontal line “gate”.

FIG. 3 illustrates a representative sample obtained from a patient at high risk for developing lung cancer. The first two profiles in the upper panel (FIG. 3A and FIG. 3B) show the light-scatter gates to exclude debris and BECs, respectively. An additional doublet discrimination gate that excludes cell doublets (FIG. 3C) was applied as well. The cells that fall within the diagonal box are single cells (SC). The upper most right profile (FIG. 3D) shows the cells selected through the previous three light-scatter gates (eliminating debris, BECs and cell doublets), stained with a PE-labeled isotype control antibody to determine the background staining for the PE-labeled CD45 antibody. The specific CD45-PE staining in this sample is depicted in FIG. 3E, where the CD45^{positive} cells are identified with the upper box. cD45^{positive} population of sputum cells co-stained with the FITC-labeled isotype control antibody is illustrated in FIG. 3F and the FITC-labeled CD66b antibody is illustrated in FIG. 3G. The CD66b^{positive} cells are indicated by the upper box in FIG. 3G. To confirm that these cells are granulocytes, CD45^{positive}CD66b^{positive} cells were sorted using the FACSAria instrument, transferred to a slide by cytocentrifugation and stained with Wright-Giemsa. As shown in FIG. 3H, the blood cells that were identified with the CD66b^{positive} antibody were indeed granulocytes.

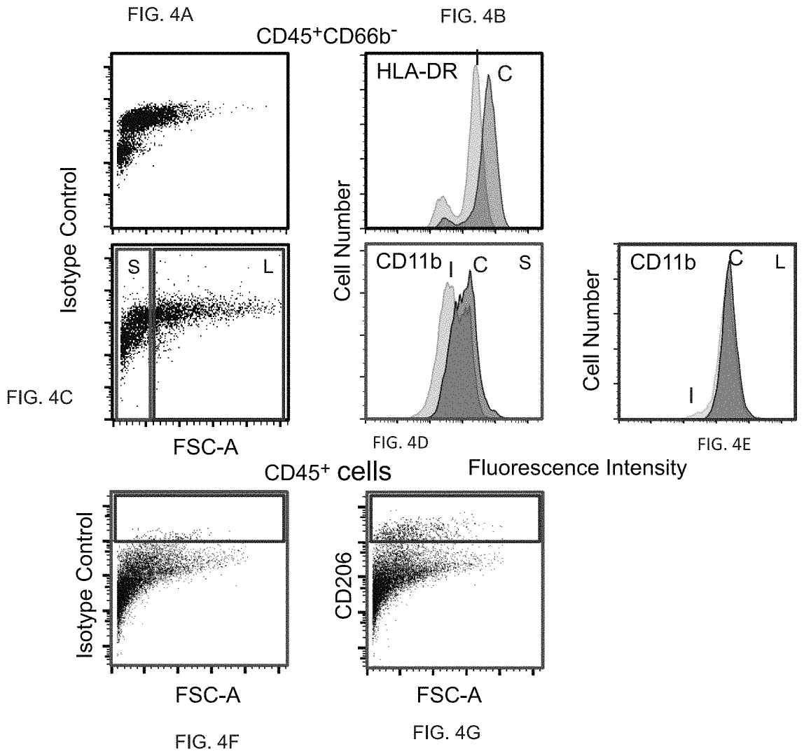

The remaining CD45^{positive}CD66b^{negative} cells can include all other types of hematopoietic cells, but are most likely macrophages and monocytes, or lymphocytes, since other hematopoietic cells in sputum are relatively rare (17,27). Specific markers for macrophages confirmed that the majority of the cell population in FIG. 4A are CD45^{positive}CD66b^{negative} macrophages/monocytes since they expressed HLA-DR and/or CD11 b.

Referring now to FIG. 4 A-G, CD45^{positive} sputum cells exposed to either CD66b probe or CD206 probe are illustrated. FIG. 4A-E illustrate a CD66b^{negative} population that includes a variety of macrophage populations. FIG. 4A CD45^{positive}CD66b^{negative} sputum cells express HLA-DR and in some cases CD11 b. FIG. 4A illustrates a dot plot showing CD45^{positive}CD66b^{negative} sputum cells stained with an isotype control to determine the background staining for the anti-HLA antibody. The same isotype control staining is also represented in the histogram at FIG. 4B by the light-gray curve (I). The dark-gray curve in FIG. 4B represents the HLA-DR staining of the same cells (C). The right shift of the dark-gray curve compared to the light-gray curve indicates that the cells stain positive for HLA-DR. The isotype control for determining the background staining for the anti-CD11b antibody is presented in FIG. 4C. The CD45^{positive}CD66b^{negative} cell population was divided into small (S) and large (L) cells so that the CD11 b staining could be better visualized in the fluorescence histograms in FIG. 4D and FIG. 4E respectively. The isotype control (I) is represented by the light gray curves in the “S” and “L” histogram, while the anti-CD11 b antibody staining (C) is depicted by the dark-gray curve in the “S” and “L” histogram. Only the small cells stain positive for CD11b. FIG. 4F-G illustrate an isotype control (dot plot on the left) and CD206 staining (dot plot on the right) of CD45^{positive} sputum cells. FIG. 4 A-B illustrate D45^{positive}CD66b^{negative} sputum cells which include a variety of macrophage populations. FIG. 4A CD45^{positive}CD66b^{negative} sputum cells express HLA-DR epitope and in some cases CD11 b. The CD11 b marker is found on myeloid cells.

In another embodiment, combining the CD3/CD19 markers with the CD66b marker allows identification of potential lymphocyte contamination in the macrophage/monocyte population (the CD66b^{negative}/CD3^{negative}/CD19^{negative} subset of cells) in those samples that happen to harbor a discernible lymphocyte population (28-30). Gating the CD3^{positive}/CD19^{positive}/CD66b^{positive} population of cells out of the CD45^{positive} population of cells analyzed for TCPP signal is yet another method for improving signal related to the TCPP label.

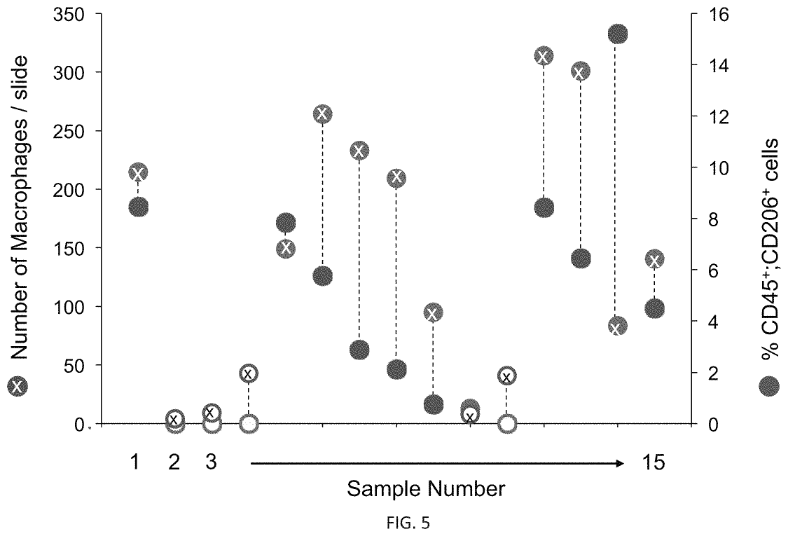

Referring now to FIG. 5, the presence of a CD206^{positive} cell population that coincides with the presence of numerous macrophages on a sputum smear is illustrated. Fifteen sputum samples were independently analyzed for the presence of macrophages by a Wright-Giemsa-stained sputum smear and CD206 staining on a flow cytometer. It should be noted that the Wright-Giemsa staining of the sputum smear can be substituted by a PAP staining. Plotted are the number of macrophages counted per slide (solid dots with x) and the % of CD45^{positive}CD206^{positive} cells (solid dots) for each of the fifteen samples analyzed. The dotted black lines are added to indicate that the data represents the same sample. The absence of macrophages on the slides is represented by the open white dots, and an inconclusive CD206 profile is represented by an open white dot with x. As shown in FIG. 5, when an abundance of macrophages is identified on a sputum smear, a distinct population of CD45^{positive}CD206^{positive} cells is also observed by flow cytometry. When there are no or few macrophages on the slide, the CD45^{positive}CD206^{positive} profile is not reliable. The presence of a well-defined population of CD45^{positive}CD206^{positive} cells in sputum (irrespective of size) coincides with a large number of macrophages observed on the slide (>13), indicating a high quality (i.e., deep-lung) sputum sample. If there is no CD45^{positive}CD206^{positive} cell population present (samples 2, 10 and 11) or it is hard to recognize (samples 3 and 4), the sputum smear shows 0 to few macrophages (≤13), indicating this sputum sample is of inferior quality. Fifteen sputum samples were independently analyzed for the presence of macrophages by a Wright-Giemsa-stained sputum smear and CD206 staining on a flow cytometer. Plotted are the number of macrophages counted per slide (solid dots with an X) and the percentage (%) of CD45^{positive}CD206^{positive} cells (solid dots) for each of the fifteen samples analyzed. The dotted black lines are added to indicate that the data represent the same sample. The absence of macrophages on the slides is represented by the open dots, and an inconclusive CD206 profile is represented by an open dot with an X.

### Identifying Cancer Cells in Sputum by the CyPath® Assay

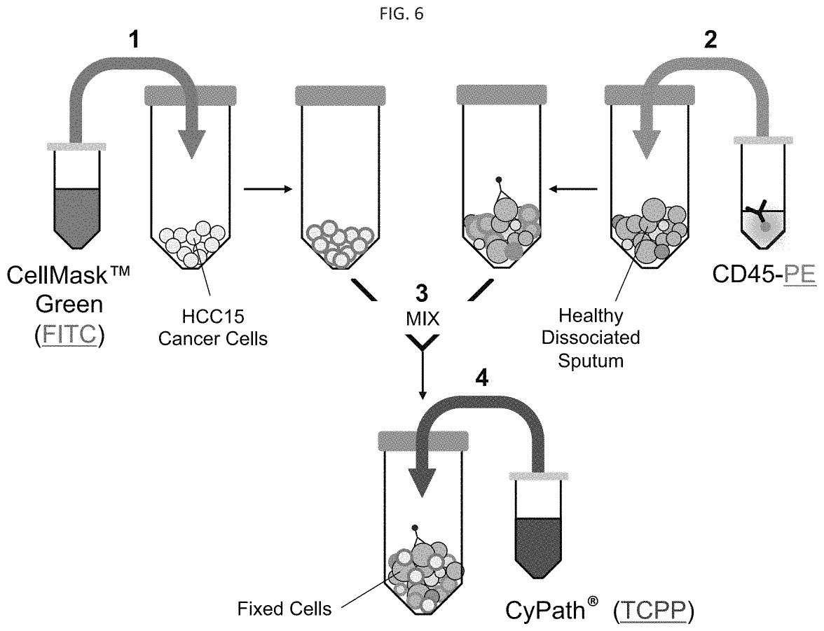

Another component of the flow cytometry-based sputum analysis for early cancer detection is the CyPath® labeling of cancer cells. We analyzed sputum samples obtained from high-risk patients (presumably without lung cancer) and spiked the sample with approximately 3% HCC15 cancer cells. For this experiment, which is outlined in FIG. 6, HCC15 lung cancer cells were labeled with CellMask™ Green so that all cancer cells could be identified in the mixture by this green color. The sputum cells were stained with an anti-CD45-PE antibody, so that we could distinguish hematopoietic cells from non-hematopoietic cells, including HCC15 cells which are CD45^{negative} (data not shown). After cell fixation, the cell mixture was labeled with TCPP, and the cells were analyzed by flow cytometry.

Referring to FIG. 6, experimental set up of sputum analysis spiked in with lung cancer cells is illustrated. HCC15 cancer cells were labeled with CellMask™ Green (step 1) while, in a different tube, dissociated sputum cells were stained with a PE-labeled anti-CD45 antibody (step 2). After washing out the excess CellMask™ Green and the anti-CD45 antibody of the respective tubes, the two cell suspensions were mixed (step 3). The mixed cell suspension was then fixed and incubated with the CyPath® solution, which carries TCPP as the fluorescent ingredient (step 4). FIG. 6, a flow chart of sputum sample preparation for analysis, is illustrated. HCC15 cancer cells were labeled with CellMask™ Green (step 1) while, in a different tube, dissociated sputum cells were stained with a PE-labeled anti-CD45 antibody (step 2). After washing out the excess CellMask™ Green and the anti-CD45 antibody of the respective tubes, the two cell suspensions were mixed (step 3). The mixed cell suspension was then fixed and incubated with the CyPath® Assay solution, which carries TCPP as the fluorescent ingredient (step 4).

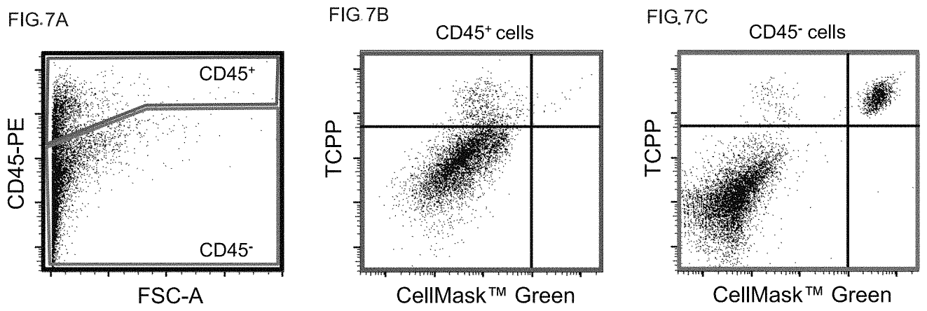

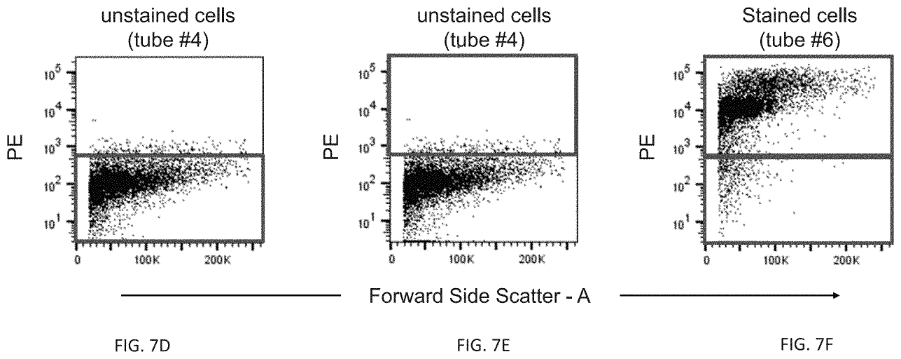

Referring now to FIG. 7A-C, dot plots of sputum cells treated with CD45-PE marker, cell mask green and TCPP are illustrated, wherein the sample was spiked in with lung cancer cells (HCC15). FIG. 7A is a representative dot plot of CD45 expression on sputum cells spiked in with ˜4% HCC15 lung cancer cells. The HCC15 cells (CD45^{negative}) were previously labeled with the green fluorescent dye CellMask™ Green (see FIG. 6). The upper gate indicating the CD45^{positive} cells is based on the appropriate isotype control (see FIG. 7D). The bottom gate indicates the non-hematopoietic, CD45^{negative} cells. FIG. 7B illustrates a dot plot analysis of CD45^{positive} cells for TCPP (y-axis) and CellMask™ Green staining (x-axis). A clearly identifiable population of CD45^{positive} cells, most likely macrophages stained positive for TCPP and are in the upper-left box. FIG. 7C illustrates a dot-plot analysis of CD45^{negative} cells for TCPP (y-axis) and CellMask™ Green staining (x-axis). The CellMask™ Green^{positive} cells are the HCC15 cells added to the sputum sample and all stain positive for TCPP (upper-right quadrant). The CellMask™ Green^{negative} cells are the sputum cells, showing a background staining of 1.2% (lower left quadrant). After the three light-scatter gates shown in FIG. 7 A-C were applied to the mixture of sputum cells and HCC15 cells, cells were analyzed for CD45 expression (FIG. 7A). TCPP uptake was then determined in both the CD45^{positive} (population outlined with the upper box) and the CD45^{negative} cell population (population outlined with the lower box). Only a small population of CD45^{positive} cells show TCPP uptake (FIG. 7B). In contrast, the CD45^{negative} cells show a very discrete population of TCPP^{positive} cells, which also stain positive for CellMask™ Green (FIG. 7C upper-right quadrant). Since the only cells treated with CellMask™ Green are the HCC15 lung cancer cells, the TCPP^{positive} CellMask™ Green^{positive} cells are the spiked in HCC15 lung cancer cells. There were no CellMask™ Green^{positive} cells that did not stain with TCPP (FIG. 7C, lower-right quadrant), indicating that CyPath® stained all cancer cells spiked into the sputum sample.

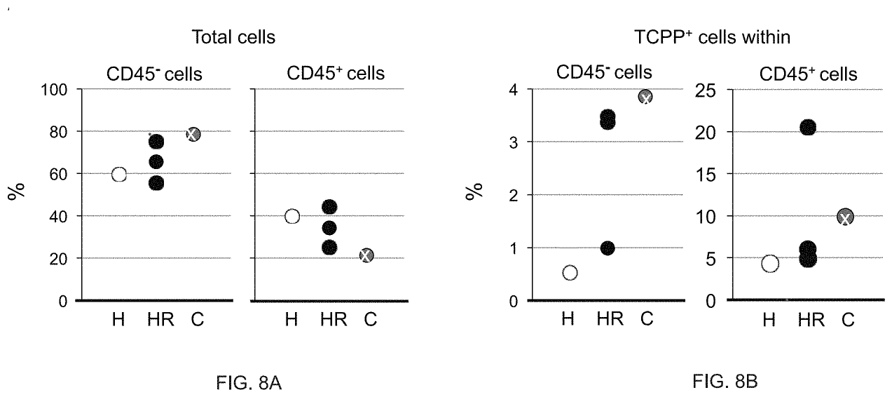

Five sputum samples in a small pilot experiment were analyzed: One sample from a healthy volunteer, three samples from high risk patients without cancer and one sample from a lung cancer patient. The analysis was performed as described in FIG. 7, meaning that each sample was spiked in with CellMask™ Green-labeled HCC15 cells and analyzed as described for FIG. 7. The rationale for spiking the samples with HCC15 cells is that these cells would serve as a positive control for the CyPath® stain. Although there was only one C sample among the five samples analyzed, the data suggest that a sputum sample from a lung cancer patient is different from that obtained from another patient without the disease: the C sputum sample contained more CD45^{negative} cells and fewer CD45^{positive} cells than the samples harvested from individuals without cancer (FIG. 8A). Most important, the C sample displayed the highest number of TCPP^{positive} cells among the CD45^{negative} (epithelial) cell population. TCPP labeling in the CD45^{positive} population did not uniquely identify the C sample from the other, non-cancer samples (FIG. 8B).

Referring now to FIG. 8 A-B, a preliminary, comparative analysis of sputum samples obtained from healthy volunteers and high-risk patients with and without cancer is illustrated. Five samples from different donors were analyzed, similar to the experiment detailed in FIG. 6 and FIG. 7. The open dots represent a sample from a healthy volunteer (H), the black dots samples represent a sample from a high-risk patient without cancer (HR) and the dot with x represents a sample from a confirmed lung cancer patient (C). FIG. 8A illustrates the total numbers of CD45^{negative} (left) and CD45^{positive} cells (right) within each sample analyzed. FIG. 8B illustrates the proportion of TCPP^{positive} cells within the CD45^{negative} (left) and CD45^{positive} cells (right) within each sample analyzed.

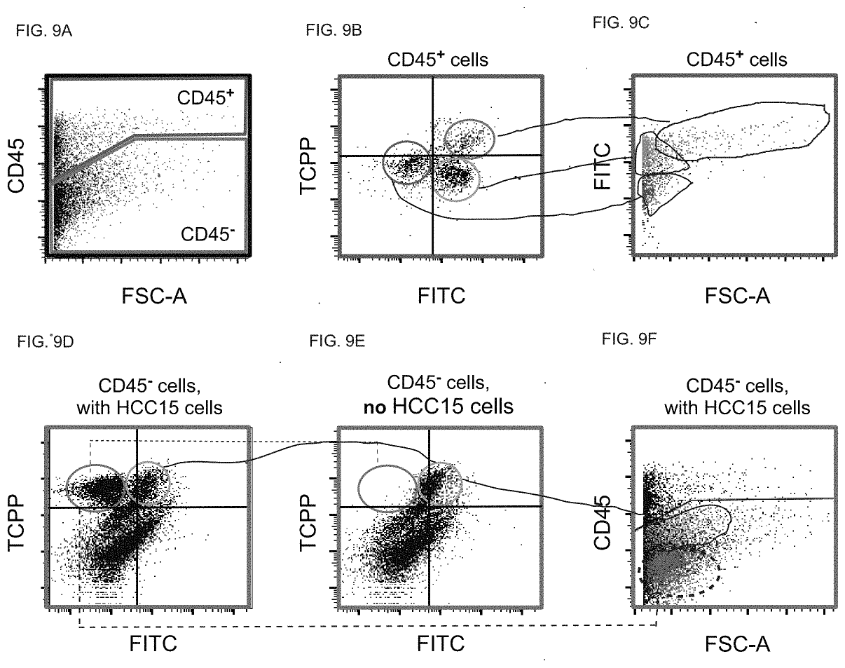

Referring now to FIG. 9 A-F, one strategy for analyzing sputum cells for the presence of TCPP^{positive} cells is illustrated according to one embodiment of the present invention. FIG. 9A illustrates a dot plot of a mixture of sputum cells with HCC15 cells mixed therein are treated with an anti-CD45-PE antibody. The upper gate includes the CD45^{positive} cells and is based on the appropriate isotype control (not shown). The lower gate indicates the non-hematopoietic, CD45^{negative} cells. FIG. 9B depicts cells treated with TCPP and a cocktail of FITC-labeled probes. The FITC-labeled probes include antibodies directed against CD66b (granulocytes), CD3 and CD19 (lymphocytes). FIG. 9B has four quadrants: The cells above the horizontal line are cells that stained positive for TCPP, while the cells to the right of the vertical line are cells that are stained positive for FITC. The circles are drawn to indicate the different cell populations present in this sample. FIG. 9C represents analysis of the same cells as in FIG. 9B, depicted in a dot plot showing FITC intensity (y-axis) vs. FSC-A (x-axis; representing cell size). Cell populations are identified between FIG. 9B and FIG. 9C. The cells from the lower-right quadrant show a profile consistent with granulocytes, while the cells from the upper-right quadrant in FIG. 9B show a profile consistent with that of alveolar macrophages. FIG. 9D illustrates the TCPP labeling (y-axis) vs. FITC fluorescence intensity (x-axis) of CD45^{negative} sputum cells including the HCC15 cells that are spiked into the sample. Since the CD45^{negative} fraction of sputum cells includes the HCC15 cells, we expect to find a large population of TCPP^{positive} cells in this panel. There are two TCPP^{positive} populations in this sample, as indicated by the circle on the upper left quadrant and the circle on the center and upper-right quadrant. FIG. 9E illustrates the profile of CD45^{negative} cells as in FIG. 9D, but from a control sample that did not include HCC15 cells spiked into the sample. The upper left quadrant cell population in FIG. 9D is absent in the dot-plot profile of FIG. 9E at the upper left quadrant (empty circle). The cells missing from this empty circle are HCC15 cells. FIG. 9F represents the same cell population as in FIG. 9D, with the dot plot showing CD45-PE intensity (y-axis) vs. FSC-A (x-axis). The upper left cell population and upper-right and center cell populations in FIG. 9D and FIG. 9E are identified in FIG. 9F.

FIG. 9 suggests that the TCPP staining in CD45^{positive} cells is related to the alveolar macrophage population. The CD45^{positive} (hematopoietic) cell compartment (FIG. 9A) was subdivided into three subpopulations of cells based on the fluorescence intensity in the FITC channel and TCPP (FIG. 9B). When backgated on the CD66b/CD3/CD19 vs. FSC profile, the population indicated by the lower-right population of circled cells in FIG. 9B that did not stain with TCPP, appeared to be relatively small cells that stained positive with the CD66b/CD3/CD19 cocktail (FIG. 9C); these cells are likely granulocytes. The other FITC-positive population in FIG. 9B, (upper-right circled cell population and staining positive for TCPP) turn out to be relatively large cells. Their green-fluorescence is most likely due to autofluorescence and not due to CD66/CD3/CD19 staining as shown earlier by the isotype control profile in FIG. 3F. The large size and high autofluorescence suggest that the cell population in the upper right are likely alveolar macrophages (35, 36). The lower left cell population in FIG. 9B consists of relatively small cells, and, because this subpopulation is also CD66/CD3/CD19^{negative}, is likely the cell population of a different subset of macrophages and/or monocytes. CD45^{negative} cells were similarly analyzed (FIG. 9C-E). Here we compared the HCC15 cells added to the sample with an aliquot that did not include added spiked-in HCC15 cells, but was otherwise treated similarly (compare FIGS. 9C and 9D). The population that is absent in the sample without spiked-in HCC15 lung cancer cells are encircled. The cells, which stain positive for TCPP, are medium-size cells that do not express CD45 and are absent in FIG. 9E as represented by upper left empty circle. The absence of a cell population in the upper left circle for a sample not containing HCC15 confirms the TCPP staining profile of HCC15 cell population (FIG. 9E). The other TCPP^{positive} cell population among the CD45^{negative} sputum cells (encircled in center/upper right) includes cells of similar size as HCC15 cells (FIG. 9F). These cells are also CD45^{negative} but they can be distinguished from HCC15 cells by low levels of autofluorescence in the FITC channel (FIG. 9D and FIG. 9E).

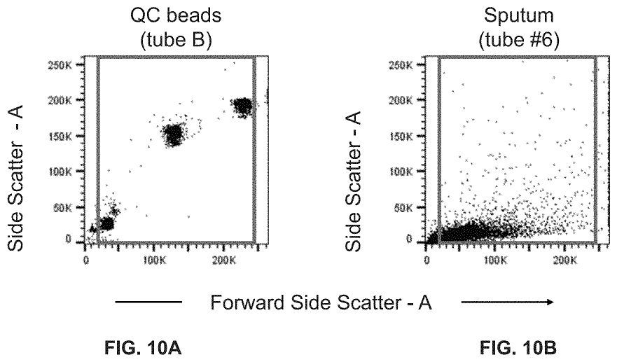

Referring now to FIG. 10A-B, quality control (QC) beads are used to establish the bead-size-exclusion (BSE) gate in the dot plot of FIG. 10B. The sputum sample in FIG. 10B is gated to remove from analysis those cells that fall to the left of the gate positioned around about 5 μm bead size and to the right of the gate positioned around 30 um bead size. The sputum samples, controls, isotype controls, and beads are prepared as described below in EXPERIMENTAL PROTOCOL.

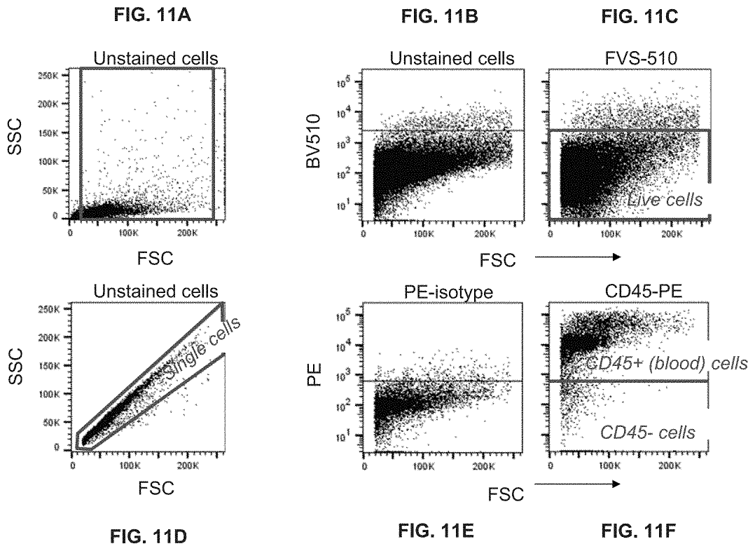

Referring now to FIG. 11A-F, treated and untreated sputum samples are analyzed via flow cytometry and the resulting dot plots are illustrated. The untreated sputum cells are first gated for size using a BSE gate to select cells that are about greater than 5 um and about less than 30 um in size for further analysis. FIG. 11A illustrates a dot plot of sputum cells that fall within the size range. The size gate is referred to as BSE gate. The BSE gate excludes debris and erythrocytes, but not squamous epithelial cells (SECs). Since SECs are dead, they will be eliminated from the sputum sample analysis with the viability dye FVS510. FIG. 11B-C illustrate dot plots of sputum cells that are untreated (FIG. 11B) and treated (FIG. 11C) with BV510 fluorescence vs. Forward Side Scatter. Sputum cells that do not take up the dye are live cells (LC) and are located below the line in FIG. 11C. The live cell gate is referred to as LC gate. The dye will stain the dead cells; the live cells are the cells that do not stain with FVS510. While the present example used dye FVS520, other viability stains/dyes will also work to distinguish the LC population. The threshold above which cells are considered positive for FVS510 (and thus dead) is based on the unstained control (FIG. 11B). The majority of cells (95% or more) of the unstained control should fall in the LC gate and less than 5% of the cells (“background staining”) should fall outside the LC gate. When this LC gate is then applied to the sputum samples that were stained with FVS510, the live cells are the cells inside the LC gate and the dead cells fall outside the gate.

FIG. 11D is a dot plot of an unstained sputum sample to identify single cells vs. doublet cells. Cell doublets are considered by the flow cytometer as one event and the one event may contain amounts of TCPP representative of two or more cells. Doublets can therefore create events with artificially high TCPP content and give the incorrect suggestion of being cancer cells or cancer-associated cells since TCPP is used as a marker for cancer cells. To eliminate doublets, a gate is drawn to identify a single cell (SC) population. A FSC-A vs. FSC-H dot-plot sputum cell profile is created from acquisition and the BSE/LC gates are applied for analysis of the SC population. Two diagonal straight lines are drawn along the main population's axes: one along the top (indicated as “top diagonal” in FIG. 11D and one on the bottom (“bottom diagonal”)). The bottom diagonal runs somewhat parallel to the top one and is best started from the “notch” in the population, from where cells seem to spread away from the main population, to the right (not shown). The cells that are spread out (i.e., those cells or dots that don't follow the diagonal population, are the doublets and need to be excluded from the analysis. The SC gate will only include the cells that form the diagonally-oriented population. SC cells are illustrated in FIG. 11D within the diagonal gate. The SC gate is created by connecting two diagonals: one that goes along the top of the main population (indicated by “top diagonal”) and one that follows the main population on the bottom (“bottom diagonal”). For placement of the bottom diagonal, one needs to spot a “notch” in the dot plot, which indicates the start of cells that do not follow the main, diagonally-oriented cell population. Below and to the right of the bottom diagonal (the light-gray area) includes the cell doublets that will be excluded from the SC gate. The bottom diagonal needs to cross the notch while following the main diagonal population up and downward.

FIG. 11E-F illustrate dot plots of sputum cells treated with either a PE control or a CD45 probe conjugated to a PE fluorophore. FIG. 11E is the isotype control. FIG. 11F identifies cells as either cD45^{positive} (blood cells) or CD45^{negative} (non-blood cells) and is referred to as the CD45 gate.

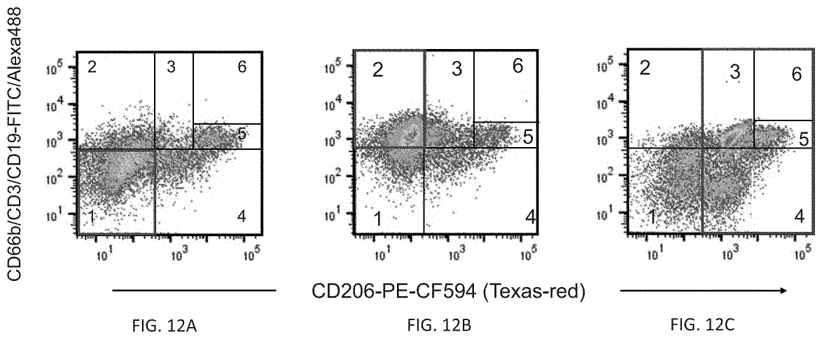

A first sputum sample from the subject is treated with a CD45 probe conjugated to a fluorophore and a cocktail of CD66B, CD3, CD19 conjugated to a fluorophore and CD206 conjugated to a fluorophore and TCPP (tube #6). FIG. 12A-C illustrate dot plots of sputum cells selected by application of the BSE, LC, SC and CD45 gates to select CD45^{positive} sputum cells treated with CD66b/CD3/CD19-FITC-Alexa488 and CD206-PE-CF594 markers. Only those cells that met the criteria of the applied gates are further analyzed. Populations of cells were identified based upon the fluorophore intensity along the CD206 antibody (x axis) and CD66b/CD3/CD19 (y-axis). In each sample, 5 to 6 populations can be identified. The relative size of each population differs from sample to sample. FIG. 12A shows profile 1 where population 1 dominates. FIG. 12B shows profile 2 where population 2 dominates. FIG. 12C shows profile 3 where the CD206^{positive} (CD206+) cells dominate, i.e., populations 3 to 6. The dominant populations in each type of profile are indicated with a bolded box. Three different signatures are depicted for CD45^{positive} sputum cells. The 5-6 populations of cells are established in light of an isotype control and control sputum sample as further identified in the following figures. The presence of macrophages indicate the sample is from deep lung. TABLE 3 identifies the cell types present in each population.

TABLE 3

| | Fluorescence | | |
|---|---|---|---|
| | FITC/ | PE-CF594 | |
| Population | Alexa488 | (Texas-red) | Cell type |
| 1 | negative | negative | monocytes, macrophages, |
| | | | other blood cells |
| 2 | positive | negative | granulocytes, lymphocytes |
| 3 | positive | positive | likely macrophages |
| 4 | negative | positive | macrophages |
| 5 | positive | high | macrophages |
| 6 | high | high | macrophages |

Referring not to FIG. 13A-B, a dot plot of isotype control for FITC/ALEXA-488 and sputum cells treated with a CD66b/CD3/CD19 probes conjugated to FITC/Alexa488 is illustrated. FIG. 13A illustrates a dot plot of CD45^{positive} cells stained with the FITC/Alexa488 isotype control is displayed as FSC on the x-axis vs. the FITC/Alexa488 on the y-axis. FIG. 13B illustrates a dot plot (similar to FIG. 11A) of CD45^{positive} cells stained with a cocktail of antibodies directed against CD66b/CD3/CD19-(FITC/Alexa488) and CD206-(PE-CF594). The horizontal FITC/Alexa488 gate is set based upon the cells that are above the background staining. The negative gate in the isotype control is set to include about 95% of the cells in the isotype control wherein the positive gate is set to include about 5% or less of background. The top value of the FITC/Alexa488-negative gate in CD45⁻ cells of most samples is on average 450, ranging from 100-1000.

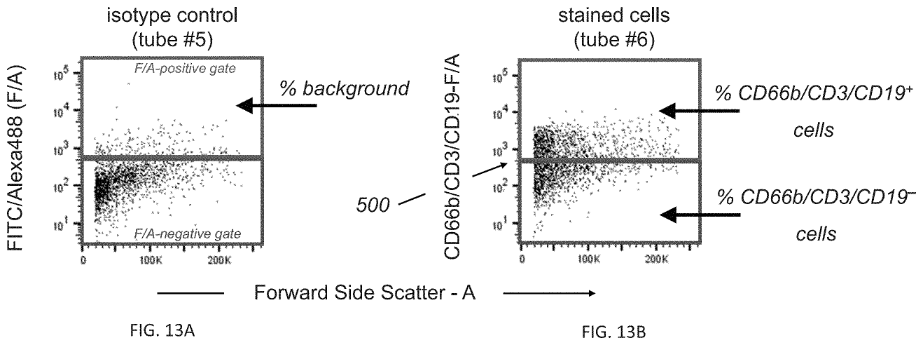

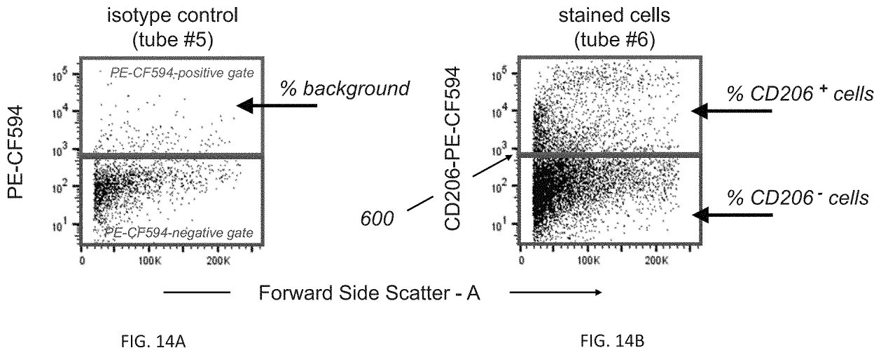

Referring now to FIG. 14A-B, a dot plot of isotype control for PE-CF594 and sputum cells treated with marker labeled with PE-CF594 is illustrated. FIG. 14A illustrates a dot plot of CD45^{positive} cells stained with the isotype controls, displayed as FSC on the x-axis vs. the PE-CF594 on the y-axis. FIG. 14B is a dot plot (similar to FIG. 14A) of CD45^{positive} cells stained with a probe/antibody conjugated to PE and directed against CD206 cell marker. FIG. 14B identifies the gate above which the population of cells positive for CD206 labeling are found. The top value of the PE-CF594-negative gate in CD45⁻ cells of most samples is on average 250, ranging from 90-500.

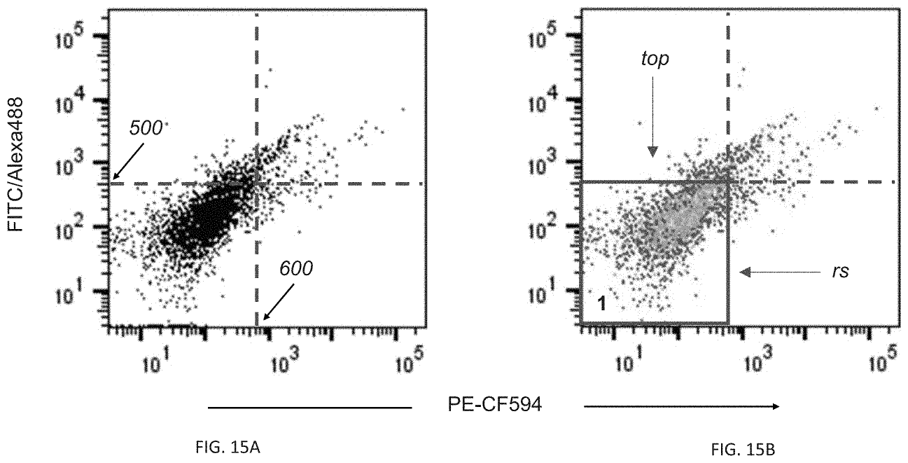

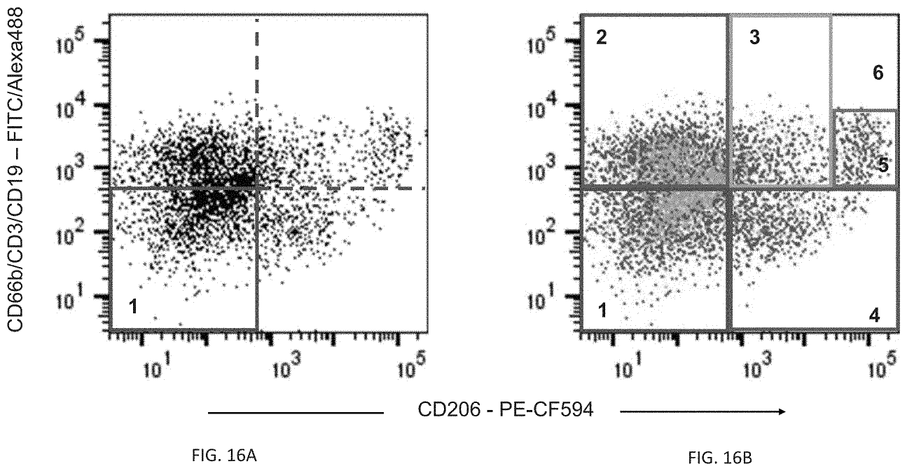

Referring now to FIG. 15A-B, a dot plot that sets the double-negative gate or population 1 is illustrated. FIG. 15A is a dot plot displaying CD45^{positive} sputum cells stained with the isotype control for the FITC/Alexa488 and PE-CF594 (Texas-Red) channels, displayed as FITC/Alexa488 on the y-axis vs. the PE-CF594 (Texas-Red) on the x-axis. FIG. 15B is the same dot plot as illustrated in FIG. 15A and illustrated as a pseudocolor plot from the isotype control, that have been gated through the BSE, LC and CD45^{positive} cell gates. The horizontal dotted line represents the FITC/Alexa488 positive/negative cut off determined in FIG. 13, whereas the vertical dotted line is derived from the PE-CF594 positive/negative cut off determined in FIG. 14. The gate for population 1, as determined in FIG. 15, is transferred to the full dot plot and pseudocolor plot of CD45^{positive} sputum cells stained with the antibodies directed against CD66b/CD3/CD19 (FITC/Alexa488—y-axis) and CD206 (PE-CF594—x-axis) as illustrated in FIGS. 16A and 16B, respectively. The top value of the FITC/Alexa488-negative gate for CD45^{positive} cells in most samples is on average 600, ranging from 200-1050. The top value of the PE-CF594-negative gate for CD45^{positive} cells in most samples is on average 500, ranging from 200-750.

Referring now to FIG. 16A-B, dot plots of a sputum sample as in FIG. 15, wherein the CD45^{positive} cells are stained with a cocktail of CD66b/CD3/CD19 antibodies conjugated to FITC/Alexa488 and CD206 conjugated with PE-CF594 and analyzed for the presence of different populations of cells. The cell populations identified as 1-5 remain after the application of the BSE, LC SC and CD45^{positive} gates. The same population 1 (box) and the cut offs (dotted lines) of FIG. 16A, are as drawn in FIG. 15 and applied to the profiles shown in FIG. 16A-B.

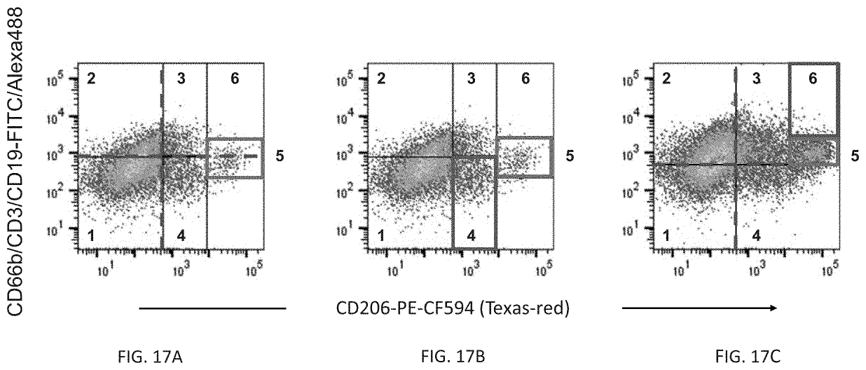

FIG. 16B illustrates the gates for populations 2-6 that are established. Populations 3, 5 and 6 are FITC autofluoroscent and should fall above the horizontal dotted line as depicted in FIG. 16A. Population 4, which is not autofluoroscent in FITC, should fall below the dotted line as depicted in FIG. 16A. Since population 2 is characterized as cells negative for CD206 (like population 1) but positive for CD66b/CD3/CD19, the gate for population 2 is drawn above population 1 and is on the right of the PE-CF594 cut off, which is the vertical dotted line FIG. 16A. The box above population 1 formed of the solid line and the dotted line is illustrated in FIG. 16B as population 2. Population 5 is identifiable as a completely isolated population on the right of the profile that is both PE-CF594^{positive} and FITC^{positive} (FIG. 16B, population 5 gate). Sometimes, population 5 is intermediate-FITC/Alexa455^{positive} and in those cases, the gate to isolate population 5 crosses the dotted horizontal red line (see FIG. 17A).

Referring now to FIG. 17A-C, pseudocolor dot plots from the sputum samples that are CD45^{positive} and treated with probes for CD66b/CD3/CD19-FITC/Alexa488 from two samples (FIG. 17A-B are the same sample but displaying different gates. All plots show CD45^{positive} sputum cells that have been gated through the BSE, LC and SC gates. The horizontal and vertical dotted lines were set on the isotype controls (not shown). FIG. 17A-B demonstrate in a drawing of gates 4 and 5, when the FITC mean fluorescence intensity of population 5 is intermediate and crossing the cut-off line. FIG. 17C illustrates a population 6 upper-right box.

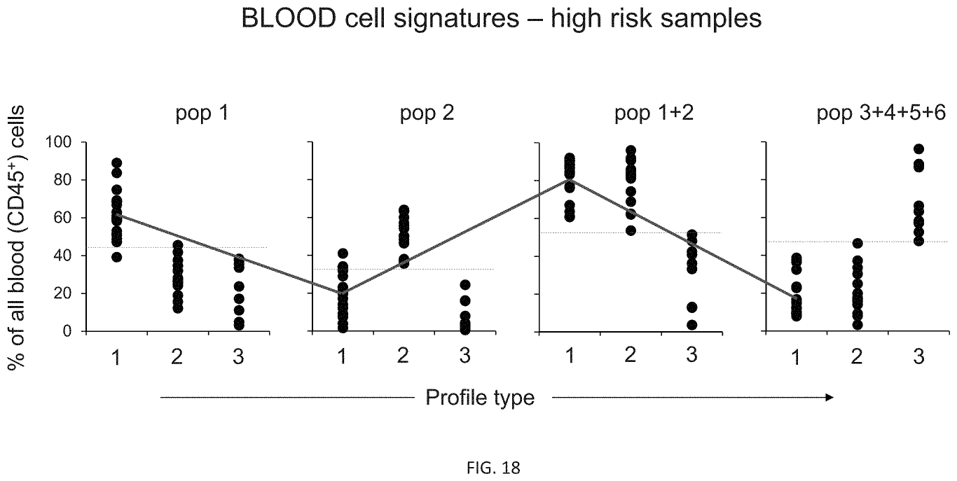

Referring now to FIG. 18, each ( ) on the x-axis reflects the profiles from FIG. 12A-C. For profile 1, the median value of each population (population 1, population 2, population 1+2, population 3+4+5+6) as a percent (%) of all CD45^{positive} cells is plotted for high risk (HR) sputum samples. The median value of each population for a profile group is connected by a straight line. A signature for profile 1 is created by drawing a line between the median value for each population identified in FIG. 18 for profile 1. A signature for profile 2 and 3 is similarly generated for sputum samples from subjects at high risk of developing lung cancer and from subjects identified as having lung cancer.

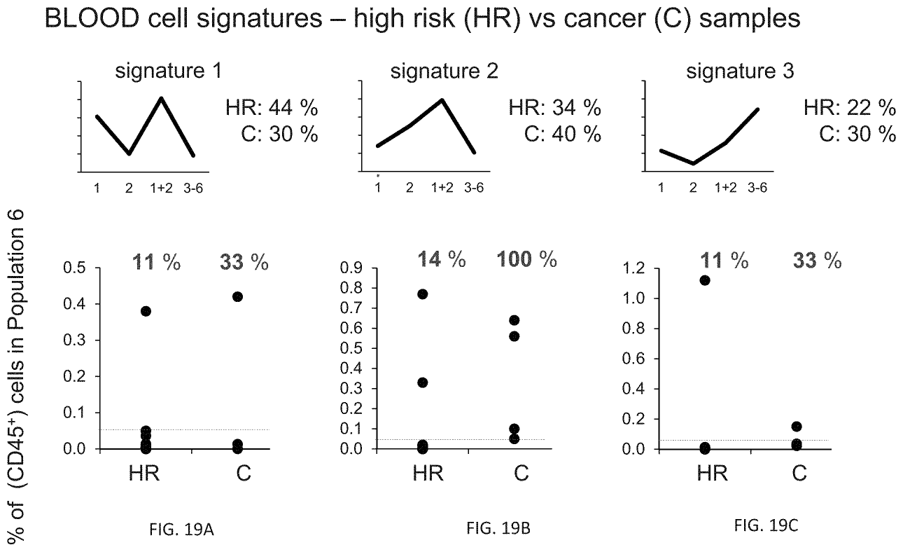

Referring now to FIG. 19A-C, a comparison of blood cell signatures from sputum collected from a subject at high risk (HR) for developing lung cancer and a subject identified as having cancer (C) is illustrated. FIG. 19A illustrates the profile 1 signature (signature 1) from FIG. 18. FIG. 19B illustrates a profile 2 signature (signature 2). FIG. 19C illustrates a profile 3 signature (signature 3). The percentage (%) of cells in population 6 was determined and identified for each signature for HR and C sputum samples.

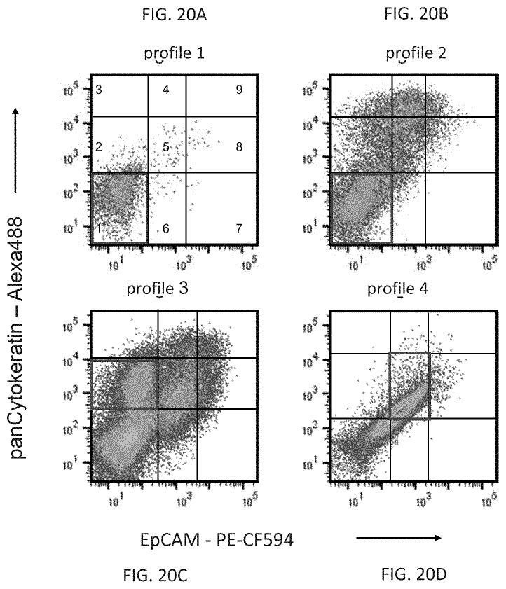

FIG. 20A-D illustrate dot plots of sputum cells that have been treated as per tube #7 with CD45 and a cocktail of panCytokeratin-Alexa488 and EpCAM-PE-CF594 data and TCPP. The cells depicted in the dot plot are those remaining after the BSE, LC, SC, CD45 gates are applied. The dot plot of cells for (CD45^{negative}) profiles 1-4 and the percent of all CD45^{negative} cells in each population that each profile 1-4 represents in addition to the relative TCPP fluorescence intensity that each population represents is further analyzed.

In each sample, 9 populations can be identified as illustrated in FIG. 20A. The same 9 populations are identified for each profile 2-4. The relative size of each subpopulation differs from sample to sample with each illustrating a different profile (profiles 1-4). FIG. 20A shows a type of profile where population 1 dominates and comprises more than 80% of all CD45^{negative} cells. FIG. 20B shows a type of profile where population 1 dominates as well, but it includes less than 80% of all CD45^{negative} cells; there is often a clear population of cells in one of the other gates. FIG. 20C shows a type of profile where there is still a large population 1 (although less than 80%), but the second-largest population is population 2. FIG. 20D shows a profile where population 5 is the most dominant population or the second-most dominant population after population 1. For each profile a different signature exists. The population that is most important for determining the type of signature is boxed in bold.

FIG. 21 A-B illustrate a dot plot of isotype control for CD45^{negative} sputum cells treated with FITC/Alexa488 or treated with panCytokeratin/Alexas488. Prior to analysis, gates for BSE, LC, SC and CD45^{negative} were applied to the population for analysis. Two profiles were generated: one displaying CD45^{negative} cells with forward side scatter-A (FSC-A) on the x-axis and FITC/Alexa488 on the y-axis (FIG. 21A) and one displaying CD45^{negative} cells with FSC-A on the x-axis and panCytokeratin/Alexa488 on the y-axis (FIG. 21B). The negative gate in each profile is set to encompass approximately 95% of the cells in the isotype control. The positive gate in each profile includes the rest of the space above the negative gate and should encompass 5% or less of background staining.

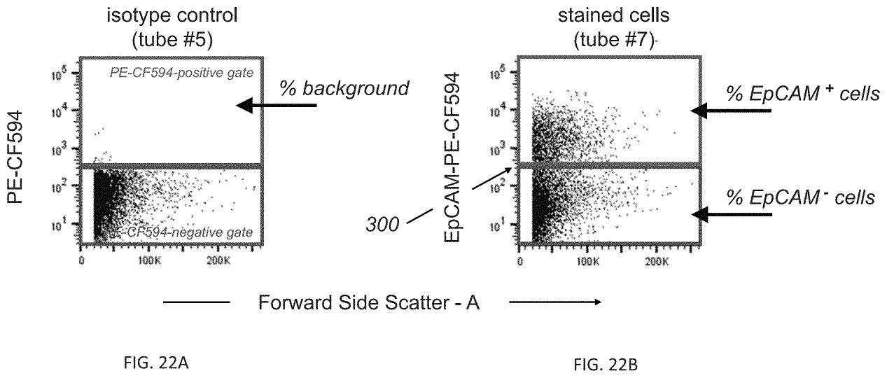

FIG. 22A-B illustrate a dot plot of isotype control for PE-CF594 and CD45^{negative} sputum cells that have been gated through the BSE, LC, SC and CD45^{negative} cell gates. Prior to analysis, gates for BSE, LC, SC and CD45^{negative} were applied to the population for analysis. Two profiles were generated: one displaying CD45^{negative} cells with forward side scatter-A (FSC-A) on the x-axis and PE-CF594 on the y-axis (FIG. 22A) and one displaying CD45^{negative} cells with FSC-A on the x-axis and EpCAM-PE-CF594 on the y-axis (FIG. 22B). The negative gate in each profile is set to encompass approximately 95% of the cells in the isotype control. The positive gate in each profile includes the rest of the space above the negative gate and should encompass 5% or less of background staining.

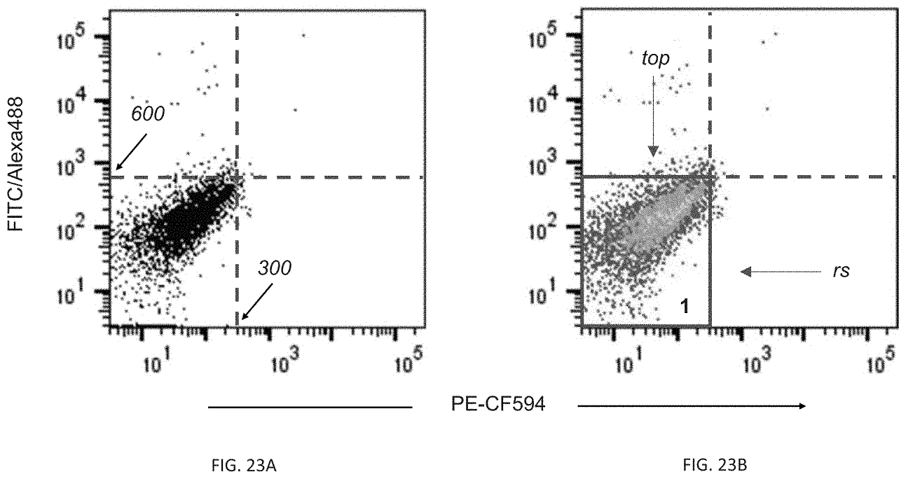

Referring now to FIG. 23A-B, a dot plot with a double-negative gate or population 1 of the CD45^{negative} cells is illustrated. FIG. 23A is a dot plot and FIG. 23B is a pseudocolor plot from the isotype control, wherein the treated sputum sample is analyzed through the flow cytometer and the events representing cells are gated through the BSE, LC, SC and CD45^{negative} cell gates. The horizontal dotted line in FIG. 23A represents the FITC/Alexa488 positive/negative cut off determined in FIG. 21, whereas the vertical dotted line is derived from the PE-CF594 positive/negative cut off determined in FIG. 22. The cut-off lines for population 1, as determined in FIG. 23, are incorporated into the full dot plot and pseudocolor plot of CD45^{negative} cells stained with the antibodies directed against all cytokeratins (Alexa488—y-axis) and EpCAM (PE-CF594—x-axis).

Referring now to FIG. 24A-B, gates for populations 2-9 of CD45^{negative} sputum cells of tube #7 are set as illustrated. FIG. 24A is a dot plot of sputum cells and FIG. 24B is a pseudocolor plot from the same sputum sample as in FIG. 23, but this time the cells are stained with an Alexa488-labeled antibody directed against all cytokeratins (y-axis) and a PE-CF594-labeled antibody directed against EpCAM (x-axis). CD45^{negative} cells are shown that were also selected through the BSE, LC and SC gates. The same population 1 (cells within solid box) and the cut offs (dotted lines extending therefrom), as drawn in FIG. 23, are applied to these profiles. Cytokeratin⁺⁺ cells indicate cells that stain highly with the panCytokeratin antibody, while the EpCAM⁺⁺ cells stain highly with the EpCAM antibody. Populations 1, 2 and 3 are EpCAM negative, so they should fall above population 1, left of the vertical, striped line that exits between population 1 and 6. The difference between the first three populations is that they express different levels of panCytokeratin. The cut off between populations 2 and 3 is determined by identifying the cells that are highly stained with panCytokeratin-Alexa488. The cut off for highly Alexa488-stained CD45^{negative} cells ranges from 10,000 to 20,000 fluorescence intensity (average 14,000), and this cut off determines the bottom line of population 3, as well as that of populations 4 and 9. FIG. 24A shows a horizontal, striped line, separating population 2 and 3 and above which cells are considered highly stained with the anti-panCytokeratin antibody in this particular sample. The cut off was determined on the pseudocolor plot, where a clear population of cells is identifiable above the 10,000-fluorescence intensity mark. Populations 1, 6 and 7 are panCytokeratin-negative, with populations 6 and 7 falling to the right of population 1, under the horizontal, striped line. The difference between populations 1, 6, and 7 is the level of EpCAM expressed on these cells. Population 7 is identified as a population of cells that highly expresses EpCAM, just like populations 8 and 9. The cut-off for cells highly expressing EpCAM is on average 3000, ranging from 1000 to 6000, The vertical, striped line in FIG. 16A indicates the cut-off for highly expressing EpCAM cells, thereby identifying the left sides of populations 7, 8, and 9. In certain embodiments, the FITC high-expressing cells will use 10,000 as the cut-off value for the PE-CF594 high-expressing cells: use 10-15× the value that identifies the top value of PE-CF594-negative gate (or the vertical, solid and striped line).

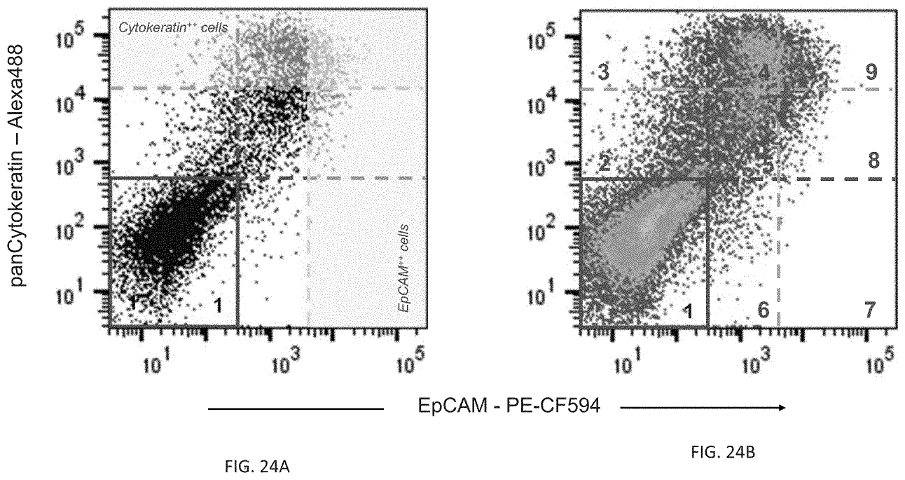

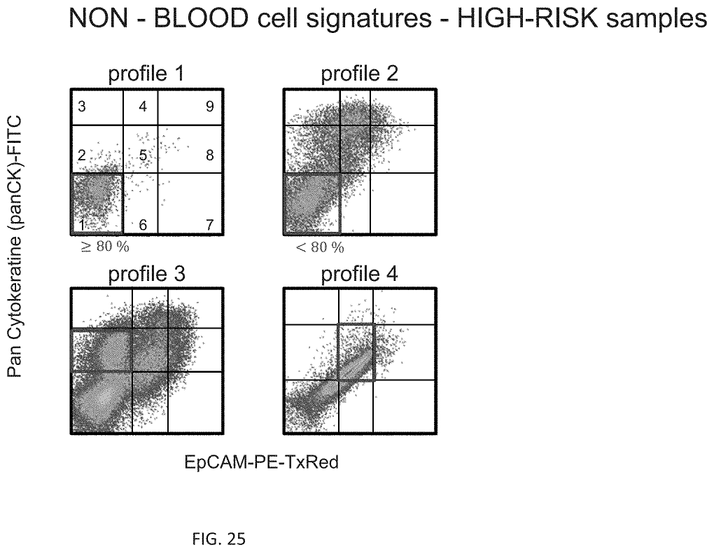

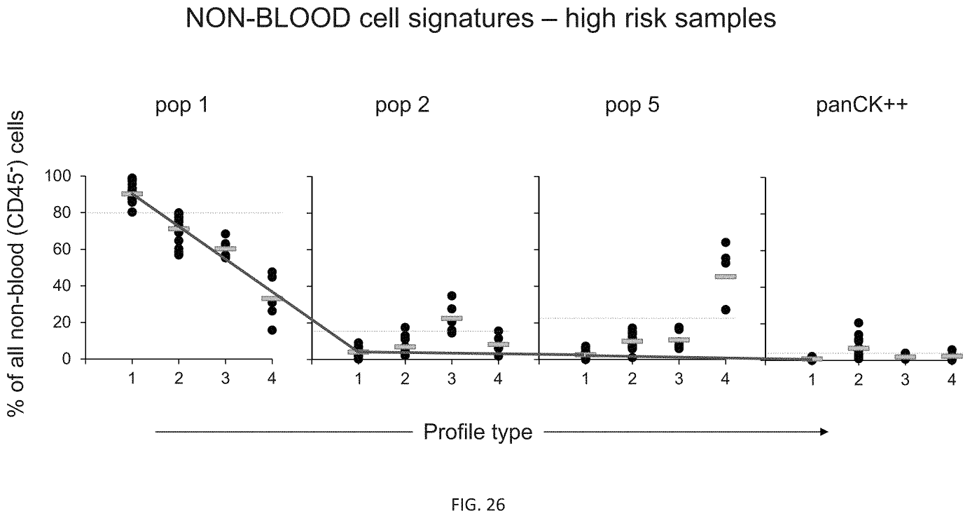

FIG. 25 illustrates dot plots of sputum cells of tube #7 from high-risk subjects remaining after the gates for BSE, LC, SC and CD45^{negative} were applied. The dot plots illustrate profiles 1-4 from subjects at high risk of developing lung cancer as shown in FIG. 20 and further analyzed in FIG. 26.

FIG. 26 illustrates a non-blood signature for profile 1 (non-blood signature 1), wherein the median value for each population (population 1, population 2, population 5 and PanCK++(CD45^{negative} in the same profile depicted in each panel is identified and a signature is generated by drawing a line from the median value for each population within a profile. A signature is generated for each profile 1-4.

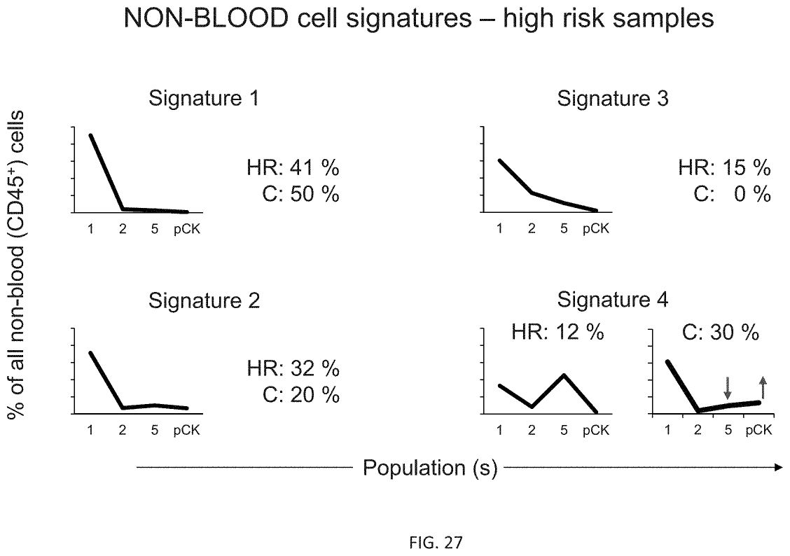

FIG. 27 illustrates non-blood signatures for sputum samples from subjects at high risk (HR) for developing lung cancer without the disease (Idct not indicative of follow up C and subjects with lung cancer (C). In Signature 4 it is noted that for the signature from C samples, the arrow at population 5 indicates a decrease in the average EpCAM cell expression while the arrow at population pCK indicates that the average panCytokeratin expression has increased as compared to the HR signature 4.

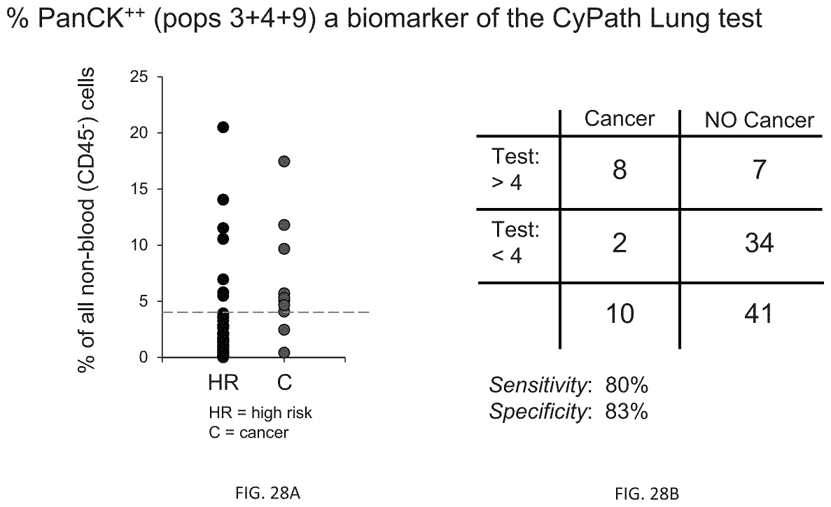

FIG. 28 A-B illustrate the sensitivity and specificity for the presence of populations 3+4+9 PanCK++ cells as a percent of all CD45^{negative} cells analyzed for sputum samples from subjects at high risk of developing lung cancer and subjects that are identified as having lung cancer. Application of the PanCK++ biomarker to the sputum samples yielded a sensitivity of 80% and a specificity of 85% for identifying cancer cells.

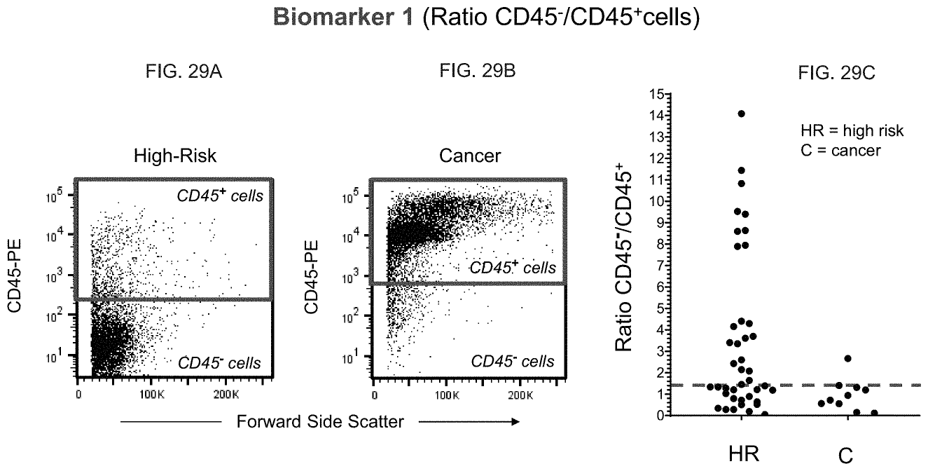

FIG. 29A-C illustrate analysis of cells in a sputum sample obtained from a subject at high risk of developing cancer and a subject with cancer after the ratio of CD45^{negative}/CD45^{Positive} (biomarker 1) cells in the sputum sample is analyzed. FIG. 29A illustrates the ratio of CD45^{negative}/CD45^{positive} cells in a sputum sample from a high-risk individual. FIG. 29B illustrates the ratio of CD45^{negative}/CD45^{positive} cells in a sputum sample from a subject that is known to have cancer. FIG. 29C is an analysis of the ratio of the CD45^{negative}/CD45^{positive} cells in the sputum sample from two subject.

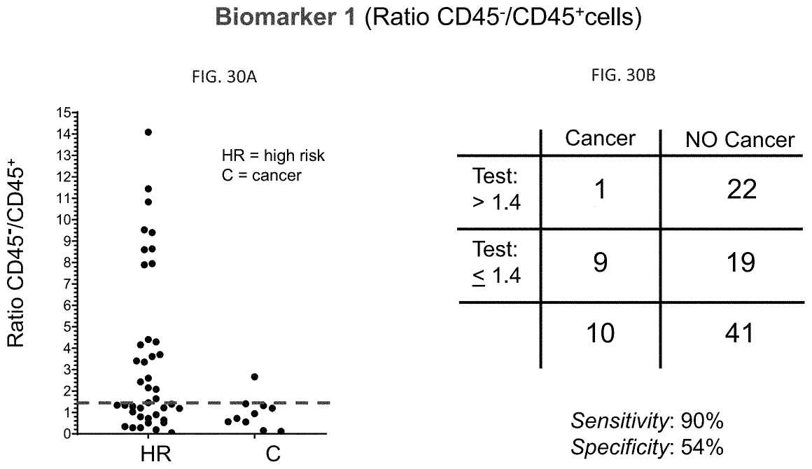

FIG. 30A-B illustrate specificity of 54% and sensitivity of 90% when the sputum sample from HR and C samples are analyzed for biomarker 1 (ratio of CD45^{negative}/CD45^{positive} cells in the sputum sample).

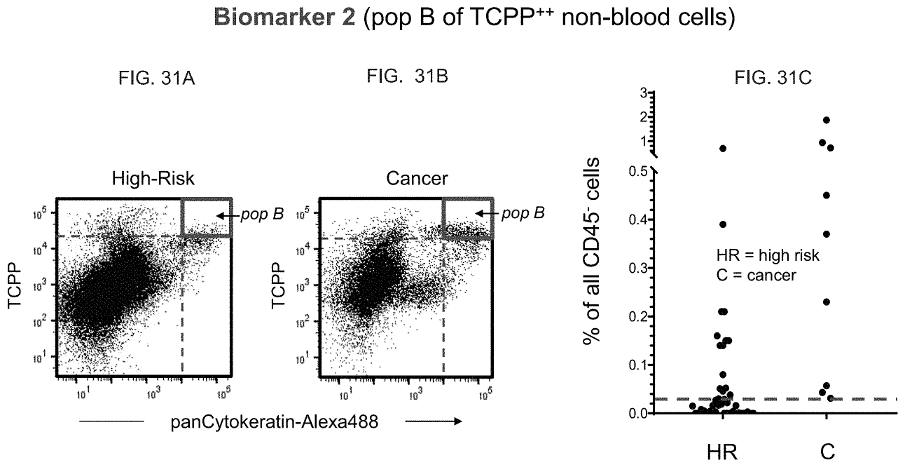

FIG. 31A-C illustrate dot plots of CD45^{negative} sputum cells of tube #7. The sputum samples were obtained from a subject at high risk of developing cancer and a subject with cancer and analyzed after the BSE, LC, SC and CD45^{negative} gates were applied. The y-axis is the TCPP fluorescence and the x-axis is the panCytokeratin-Alexa488. The presence of TCPP in cells that stain for panCytokeratin-Alexa488 in the CD45^{negative} cells in biomarker 2. FIG. 31A illustrates a dot plot of TCPP-labeled cells in a sputum sample from a high-risk individual. FIG. 31B illustrates a dot plot of TCPP-labeled cells in a sputum sample from a subject that is known to have cancer. Population B indicates the TCPP population of cells. FIG. 31C is an analysis of the percent of CD45^{negative} cells in the sputum sample that are TCPP^{positive} in population B from each subject.

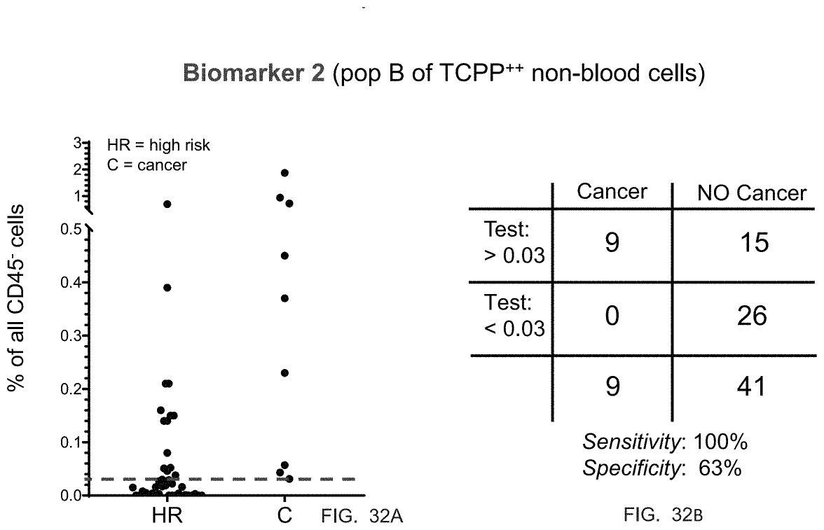

FIG. 32A-B illustrate specificity of 63% and a sensitivity of 100% for one embodiment of the method to distinguish a lung cancer (C) sputum sample from a High Risk (HR) (non-lung cancer) sputum sample with the application of biomarker 2 of FIG. 31.

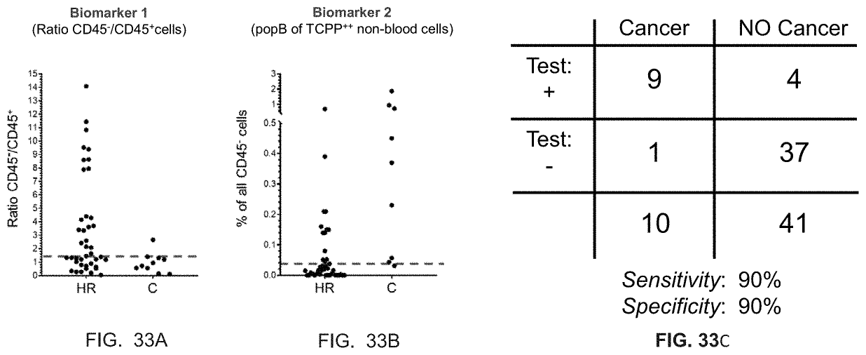

FIG. 33A-C illustrate a combination of biomarker 1 and biomarker 2 applied to the sputum sample collected as identified in FIG. 31 and FIG. 32 to analyze a sputum sample obtained from a subject that is at high risk of developing lung cancer and a subject identified as having lung cancer according to one embodiment of the present invention. FIG. 33C illustrates a sensitivity of 90% and a specificity of 90% for identifying the sample as from a subject with cancer or a subject without cancer.

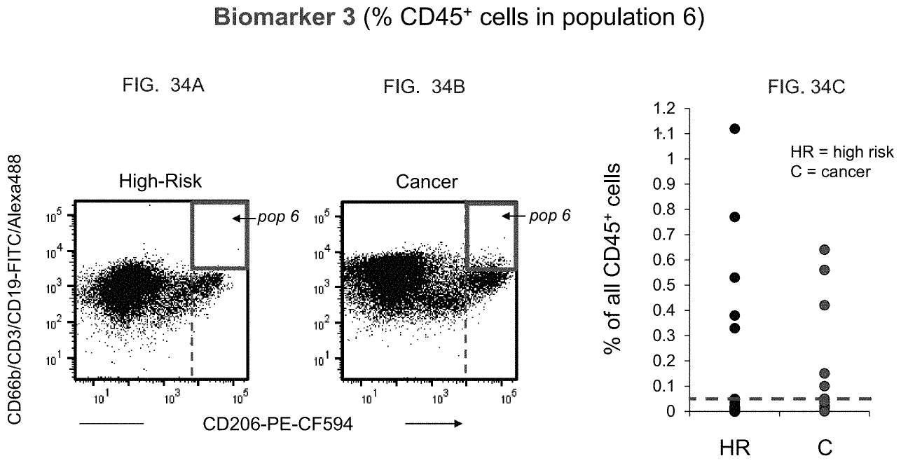

FIG. 34A-C illustrate cancer risk analysis of cells in a sputum sample labeled with CD66b/CD3/CD19 and CD206 to determine the amount of CD66b/CD3/CD19⁺⁺ and CD206⁺⁺ cells in population 6. The horizontal gate for population 6 is set at between 10,000 and 30,000 (for example, between 10,000-15,000, or 15,000-20,000, or 20,000-25,000 or 25,000-30,000) mean fluorescence intensity. The total of cells in population 6 as compared to all CD45^{positive} cells present (biomarker 3) in a sputum sample obtained from a subject that is at high risk of developing lung cancer (FIG. 34A) and a subject identified as having lung cancer (FIG. 34B) is shown in FIG. 34C.

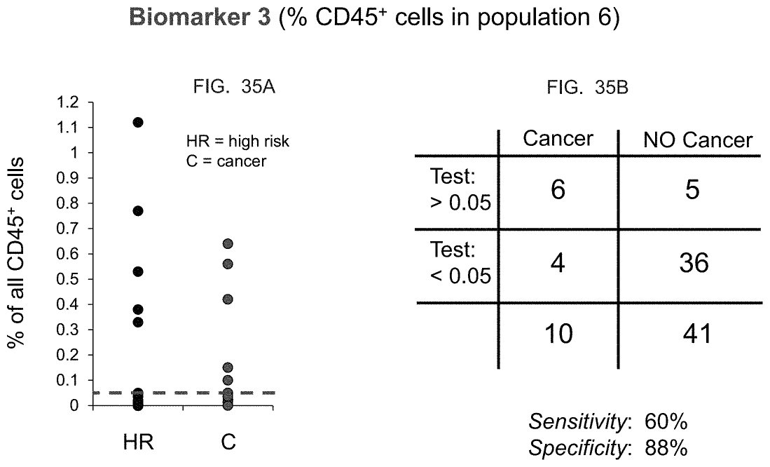

FIG. 35A-B illustrate specificity of 88% and sensitivity of 60% for one embodiment of the method to distinguish a lung cancer (C) sputum sample from a High Risk (HR) (non-lung cancer) sputum sample with the application of biomarker of FIG. 34.

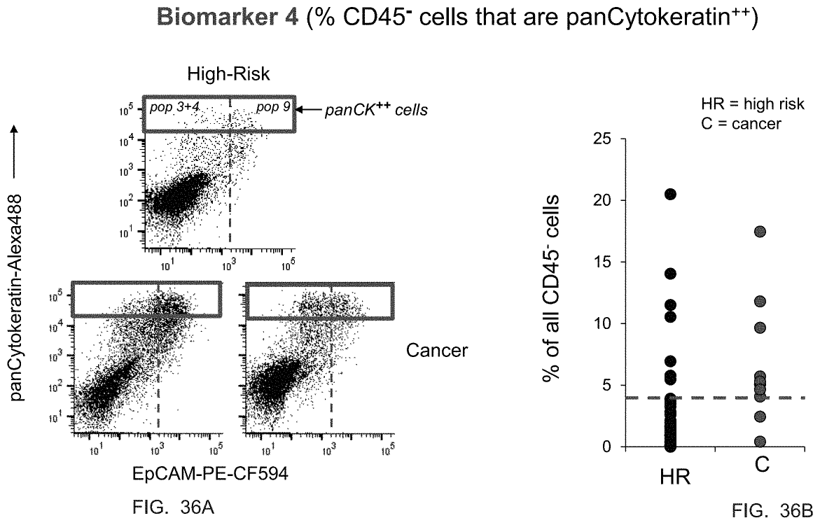

FIG. 36A-B illustrate cancer risk analysis of CD45^{negative} cells from a sputum sample collected from a subject at high risk of developing lung cancer and two subjects that are identified as having lung cancer. The percent of CD45^{negative} cells that are pancytokeratin^{positive (or high expressing)} in population 3+4+9 are identified as biomarker 4.

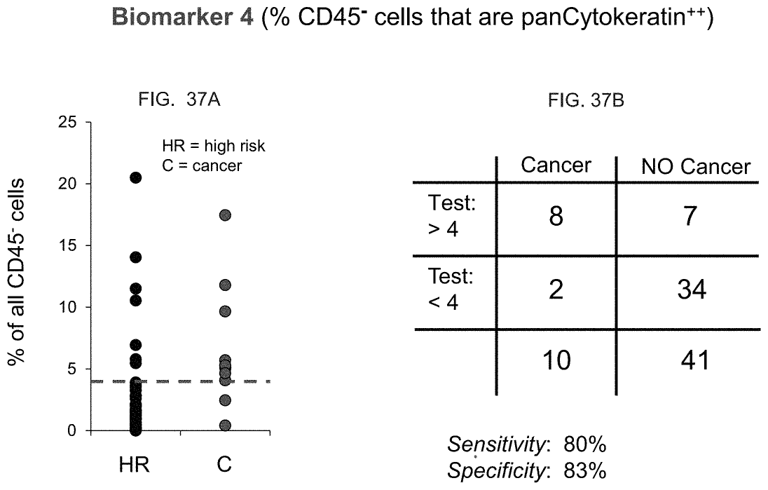

FIG. 37A-B illustrate specificity of 83% and sensitivity of 80% for one embodiment of the method to distinguish a lung cancer (C) sputum sample from a High Risk (HR) (non-lung cancer) sputum sample with the application of biomarker of FIG. 36.

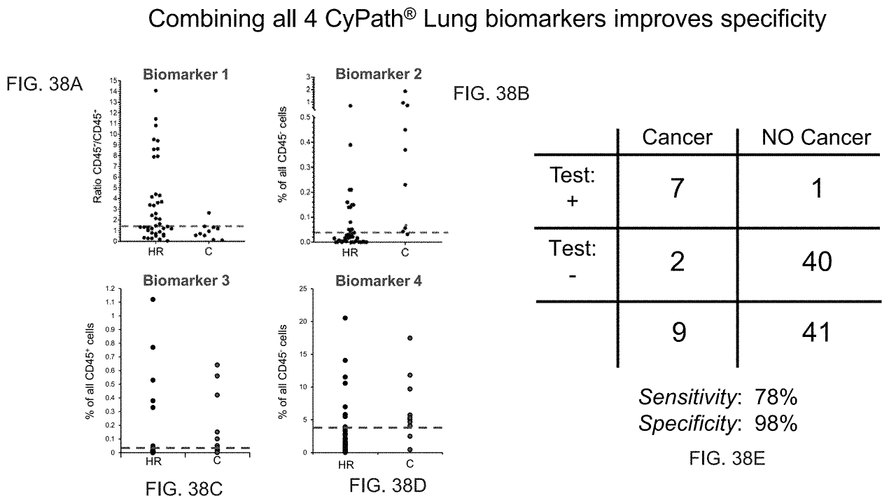

FIG. 38A-E illustrate cancer risk analysis of cells from a sputum sample from cancer and high-risk subjects with the application of a combination of biomarkers 1, 2, 3, and 4. A specificity of 98% and a sensitivity of 78% is achieved when the combination of biomarkers 1, 2, 3, and 4 are applied to the sputum samples to identify cancer samples from no cancer samples

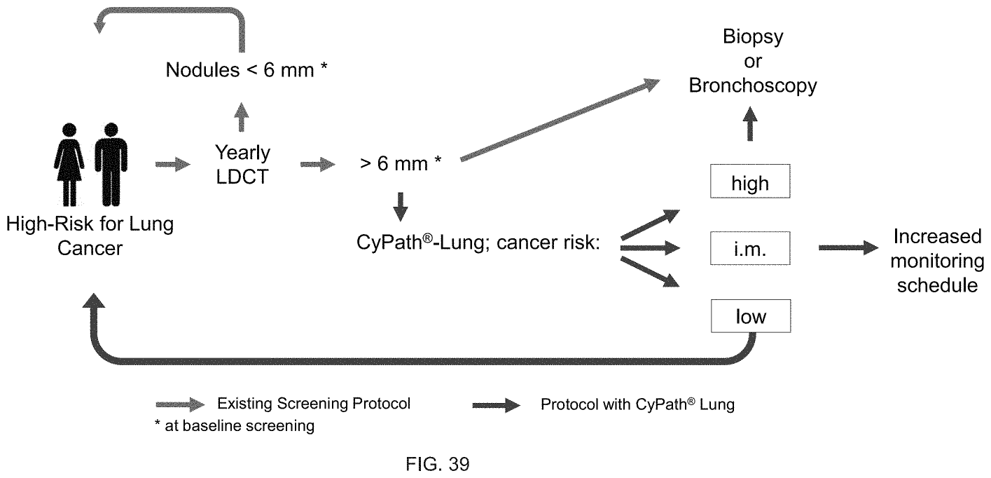

FIG. 39 illustrates a screening flow chart for lung health of subjects that include a system and method for fractionating cell populations from the lung as described herein. In a proof-of-concept clinical study with this labeling method (called the CyPath® assay), the fluorescence intensity parameter of RFCs in TCPP-labeled lung sputum combined with data on the smoking history of the patient were able to classify study participants into cancer vs. high-risk cohorts with 81% accuracy (12). Although the sensitivity of CyPath® enhanced sputum cytology was shown to be higher (77.9%) than conventional sputum cytology, the number of cells counted (600,000) from stained slides (12 slides/patient) was a limiting factor for assay sensitivity. It is predicted, using a Poisson distribution of RFCs in cancer samples, that simply doubling the number of cells for examination to >1 million could increase RFC detection to 95% (12). In addition, the need to include a separate sputum smear step for macrophage quantification to verify sample adequacy contributed to an assay design with low potential for automation or scalability. Therefore, high-throughput flow cytometry is an alternative to the slide-based testing that would support examination of millions of cellular events within a clinically relevant timeframe.

### Experimental Protocol

### Human Sputum Samples

Volunteers were recruited to provide a three-day sputum sample. Three distinct study cohorts were included: 1) individuals at high risk for developing lung cancer, but presumably cancer free, 2) high risk individuals diagnosed with lung cancer and 3) healthy individuals (22 years and older) not diagnosed with cancer and not at high risk for developing lung cancer. To be eligible for the high-risk cohort, subjects had to be heavy cigarette smokers defined as 30 pack years and between 55 and 75 years of age (13). (Examples of 30-pack-years smoking are: 1 pack per day per year for 30 years, 2 packs per day per year for 15 years, etc.) For the healthy cohort, subjects had to have smoked for ≤5 pack years and/or have quit 15 years earlier and be 22 years of age or older. Other exclusion criteria (applicable to all cohorts) were the presence of severe obstructive lung disease, uncontrolled asthma, angina with minimal exertion, pregnancy or working in the mining industry.

### Sputum Collection

All study participants were trained in the use of the Acapella® assist device (Smiths Medical, St. Paul, Minn.), in accordance with the manufacturer's instructions. The Acapella® device is an FDA-approved, hand-held device that helps to thin and mobilize mucous secretions from deep within the lung. Subjects were instructed to use the device and expel the sputum sample into a sterile collection cup. Subjects repeated this procedure at home to collect the second- and third-day sputum samples. Subjects were instructed to store their specimen cup in a cool, dark place or in a refrigerator and to return it to the site of initial collection within 1 day after collection was complete. Completed specimen cups were packed with frozen transport ice packs and sent overnight to be analyzed. The cell viability in the 3-day collection samples received (n=38) was on average 64.3% (SD: 25.6%; range: 23.6-100%), not including buccal epithelial cells (BECs or cheek cells), which are all dead (14).

### Sputum Dissociation

Sputum plugs were separated from contaminating saliva using a cotton swab (15,16). When plug selection was not possible, the whole sample was processed. The sputum was mixed with pre-warmed 0.1% dithiothreitol (DTT) at a 1:4 ratio with sputum plug weight (w/w) and 0.5% N-acetyl-L-cysteine (NAC) at a ratio of 1:1. The mixture was then rocked for 15 minutes at room temperature. GIBCO® Hank's Balanced Salt Solution (HBSS; ThermoFisher Scientific, Waltham, Mass.) was added (4 times the volume of the sputum/DTT/NAC mixture), and the resulting cell suspension was rocked for another 5 minutes at room temperature, filtered through a 40-110 μm nylon cell strainer (Falcon, Corning Inc.) to remove debris, and centrifuged at 800×g for 10 minutes. After decanting the supernatant, the cell pellet was re-suspended in 1 mL of HBSS. The total cell count was determined with a Neubauer hemocytometer using the trypan blue exclusion method to determine cell viability.

### Sputum Smears

The same cotton swabs used to transfer the sputum plugs for processing were used to transfer sputum cells onto one slide. Using an additional slide, the sputum sample was smeared between two slides to cover a large part of both slides (16). The slides were air-dried and stained with Wright-Giemsa. One or both slides were read and the number of macrophages counted by a pathologist.

### Other Human Samples

### Blood

Two 7-mL vials of peripheral blood were obtained from healthy volunteers. The majority of blood was used to obtain white blood cells (WBC) by lysing the red blood cells (RBC) with BD Pharm Lyse™ (BD Biosciences, San Jose, Calif.). The remainder was used for a source of RBC.

### Saliva

BECs were harvested from oral mucosa of healthy volunteers by scraping the inner cheek with a cell scraper. BECs-containing saliva was processed using the same protocol as that for the dissociation of sputum cells.

### Lung Cancer Cells

HCC15 lung cancer cells (ATCC, Manassas, Va.) were grown in RPMI 1640, supplemented with 10% Fetal Bovine Serum and 1% penicillin/streptomycin, in a 5% CO2 incubator set to 37° C.

### Antibodies and Reagents for Flow Cytometry

Examples of antibodies that can be used to stain sputum cells were the PE-labeled antibody directed against the pan-leukocyte cell surface marker CD45 (anti-CD45-PE), anti-CD66b-FITC to identify granulocytes, anti-CD206-FITC, anti-HLA-DR-BV421, anti-CD11b-BV650, anti-CD11b-APC and anti-CD11c-BV650 to label macrophages while anti-CD3-Alexa Fluor 488 and anti-CD19-Alexa Fluor 488 can be used to label T and B lymphocytes, respectively. Anti-CD45, anti-CD11b, anti-CD3 and anti-CD19, as well as their respective isotype controls were purchased from BioLegend (San Diego, Calif.), whereas anti-CD11c, anti-CD66b, anti-CD206, anti-HLA-DR and their respective isotype controls were purchased from BD Biosciences. Additional antibodies are listed in TABLE 2.

Tetra (4-carboxyphenyl) porphyrin (TCPP) was purchased from Frontier Scientific (Logan, Utah) and the CellMask™ Plasma Membrane Stains from ThermoFisher Scientific. Megabead NIST Traceable Particle Size Standards (5, 10, 20, 30, 40, and 50 μm) were purchased from Polysciences, Inc. (Warrington, Pa.).

All antibodies were titrated on sputum cells and in some cases on blood cells (CD3 and CD19) to determine the optimal staining concentration to reflect the largest differential in fluorescence intensity compared to their isotype controls. The optimal concentration of TCPP and EpCAM was titrated on sputum cells and HCC15 cells. The other staining reagents and beads were used as per the manufacturer's recommendation.

### Flow Cytometric Analysis and Cell Sorting

### Characterization of Sputum Cell Populations

Referring now to FIG. 1C, cells were analyzed by flow cytometry as each cell passes through a beam from a laser and the scatter of the light from the laser is detected at forward-scatter (FSC) detectors and side-scatter (SSC) detectors. The size and granularity of the cells can be characterized as is illustrated in FIG. 1E.

The cells in the sputum sample can be fractionated based upon the presence of live cells (LC) and dead cells (DC) and whether there are single cells (SC) or double cells captured as an event as described herein.

Samples of single-cell suspension of dissociated sputum samples in FIG. 2-9 were incubated with one or more of the following probes about 1 μg/mL anti CD45-PE, about 3 μg/mL anti CD66b-FITC and either anti-HLA-DR-BV421 (5 μg/mL), anti-CD11b-APC (4 μg/mL), anti-CD11c-BV650 (5 μg/mL) or a mixture of anti-CD3-Alexa Fluor 488 (2 μg/mL) and anti-CD19-Alexa Fluor 488 (2 μg/mL). In a separate tube, single-cell suspensions of dissociated sputum samples were incubated with about 1 μg/mL anti CD45-PE and 4 μg/mL anti-CD206-FITC for the determination of sputum quality. All incubations were performed on ice for 35 minutes, protected from light. After washing the cells with HBSS, cells were fixed for 30 minutes with 1% paraformaldehyde (Electron Microscopy Sciences, Hatfield, Pa.) at 4° C. Cell suspensions were then washed in cold HBSS and kept on ice until the analysis.

### TCPP/CyPath Labeling of HCC15-Spiked in Sputum Samples

Referring to FIGS. 1-9, dissociated sputum cells were labeled with the anti-CD45 antibody and fixed as described above. HCC15 cells were harvested by trypsin, washed with DPBS (ThermoFisher Scientific) and labeled with the CellMask™ Green Plasma Membrane Stain. The resulting CellMask™ Green-labeled HCC15 cells (cmgHCC15) were fixed with 1% paraformaldehyde for 30 minutes at 4° C. and washed with HBSS. Certain of the sputum cell suspensions were spiked with 3% cmgHCC15 cells. The mixture of fixed cells was then incubated with chilled TCPP (4 μg/mL) for 1 hour at 4° C. After the labeling, the cells were washed and put on ice until further analysis.

In one embodiment, samples were analyzed using a BD LSR-II flow cytometer (BD Biosciences), equipped with 4 lasers (404 nm, 488 nm, 561 nm and 633 nm). Cell sorting of whole sputum, CD45^{positive} CD206^{positive}, CD45^{positive} CD66b^{positive}, or CD45^{positive} CD66^{negative} subpopulations were performed on a BD FACSAria cell sorter (BD Biosciences). Post-collection data analysis was performed with FlowJo software (Tree Star, Inc. Ashland, Oreg.).

### Cytology

Whole sputum samples were prepared using the sputum dissociation method described above. Cytospins were prepared with 1 and 2.5×10⁵ cells per slide, using a Cytopro 7620 (Wescor, Logan, Utah) Hettich 32A (Rotofix, Beverly, Mass.) cytocentrifuges. Slides were stained with either Wright or Wright-Giemsa staining, following manufacturer's protocols. Images were produced at room temperature on a Nikon Eclipse Ti or an Olympus BX40 microscope. The Nikon microscope is equipped with an UPlanApo20×/0.7 objective and a DS-Ri2 camera, the Olympus microscope with a PLAPO60×/1.4 objective and a SD100 camera. NIS-Elements Advanced Research (Nikon) and CellSens Standard (Olympus) were used to secure the images.

Macrophages have traditionally been used to verify sputum sample adequacy. The guideline of the Papanicolaou Society of Cytopathology for evaluating sputum samples by cytology states that: “No numerical cut point for number of macrophages is consistently reported in the literature, but an adequate specimen should have numerous easily identifiable cells of this type” (31). HLA-DR and CD11 b (or CD11c), together with CD14 and CD206 have been shown to be useful markers for the flow-cytometric identification of different subsets of macrophages and monocytes within the lung (32,33). CD206 is a marker specific for alveolar macrophages that are long-lived cells, which have populated the lung during embryonic development (34). The CD206^{positive} macrophages, although of hematopoietic origin, cannot be found in the blood circulation. This population of macrophages is specific for the lung tissue (34) and is thus a good candidate to serve as a measure to verify sample adequacy.

### Sputum Sample Preparation

Samples are prepared for analysis as described in FIGS. 10-39. In brief, sputum samples are received, processed and antibody labeled and dye labeling performed on day 1. The samples are treated with TCPP and analyzed with flow cytometry on Day 2. Sputum samples analyzed in FIGS. 10-39 are treated as described below. Samples are analyzed on a flow cytometer having at least one laser, or at least two lasers, or at least three lasers and a plurality of channels, for example 5 channels or at least 5 channels but not limited thereto.

### Sputum Dissociation

Sputum samples are weighed and based upon the weight, dissociation reagents are added as follows: 1 volume of 0.5% NAC solution to sample and 4 volumes of 0.10% DTT solution to sample. The sample is vortexed and agitated at room temperature. Thereafter 4 volume of 1× Hank's Balanced Salt Solution (HBSS) based on the total current volume (sputum+NAC+DTT solution). The sample is filtered and then centrifuged at 800×g for 10 minutes. The supernatant is aspirated and the pellet resuspend with HBSS according to the sample size (for example, small (≤3 g) sample, add 250 μl HBSS, medium (>3-≤8 g) sample, add 760 μl HBSS, large (>8 g) sample, add 1460 μl HBSS). A 1:10 dilution is used for cell yield determination.

0.5% N-acetyl-L-cysteine (NAC) solution: Add 0.85 g of sodium citrate dihydrate to 45 mL of ddH₂O, 500 μL of 3 M NaOH, 0.25 g NAC and stir until dissolved. pH solution to between about 7.0-8.0 and adjust volume to 50 mL with ddH₂0

0.10% dithiothreitol (DTT) solution: Add 0.10 g DTT to 100 mL of ddH₂O and stir until dissolved. Split solution in 10 mL aliquots and freeze/store in −20° C. until use.

1 mg/mL CyPath TCPP stock solution as follows: Add 25 mL Isopropanol and 0.2 g Sodium Bicarbonate to 25 mL ddH2O and stir until dissolved. Adjust pH of solution to between about 9 to 10 if necessary. Add 0.05 g TCPP, protect solution from light, and stir until dissolved.

Table 4 indicates μl of cells to be aliquoted into tubes for counting and antibody labeling.

TABLE 4
Volume of cells (μL) to be aliquoted into
the tubes for counting and antibody labeling

Sample size

| | Small | Medium | Large |
|---|---|---|---|
| Function: | (≤3 g) | (>3-≤8 g) | (>8 g) |
| Counting | 5 | 10 | 10 |
| Labeling: | | | |
| Unstained control (#4)* | 20 | 50 | 50 |
| Isotype control (#5)* | 20 | 50 | 50 |
| Blood cell analysis (#6)* | 115 | 350 | 725 |
| Epithelial cell analysis (#7)* | 115 | 350 | 725 |

*These numbers indicate the flow cytometer tube number

Antibody/FVS Labeling

Sputum cells are aliquoted according to Table 4 to the reagents identified in Table 5 which are added to set up experimental and control tubes for the labeling of dissociated sputum cells.

TABLE 5
Labeling reagents

| | | | | | Dilu- |
|---|---|---|---|---|---|
| | | | [Stock] | [Final] | tion |
| Marker | Antibody | Clone | (μg/mL) | (μg/mL) | factor |
| Viability | Fixable Viability | — | 1000 X | | |
| Stain | Stain (FVS510) | | | | |
| Leukocytes | PE anti-human | HI30 | 10 | 1 | 1:10 |
| | CD45 (IgG1) | | | | |
| Granulocytes | FITC anti-human | 80H3 | 100 | 3 | 1:33 |
| | CD66b (IgG1) | | | | |
| T Cells | Alexa488 anti- | UCHT1 | 200 | 2 | 1:100 |
| | human CD3 | | | | |
| | (IgG1) | | | | |
| B Cells | Alexa488 anti- | HIB19 | 200 | 2 | 1:100 |
| | human CD19 | | | | |
| | (IgG1) | | | | |
| Macrophage | PE-CF594 anti- | 19.2 | 200 | 3 | 1:66.7 |
| | human CD206 | | | | |
| | (IgG1) | | | | |
| Epithelial | PE-CF594 anti- | EBA-1 | 50 | 1 | 1:50 |
| cells | human EpCAM | | | | |
| | (IgG1) | | | | |
| | Alexa488 | C-11 | 500 | 4 | 1:125 |
| | anti-human cyto- | | | | |
| | keratin (panCK) | | | | |
| panCK, CD3 | Alexa488 IgG1κ | MOPC-21 | 200 | 4 | 1:50 |
| and CD19 | | | | | |
| Isotype | | | | | |
| CD66b | FITC IgG1κ | MOPC-21 | 50 | 3 | 1:16.7 |
| Isotype | | | | | |
| CD206/ | PE-CF594 IgG1κ | X-40 | 200 | 3 | 1:66.7 |
| EpCAM | | | | | |
| Isotype | | | | | |
| CompBead | Compensation | — | — | 1 drop | |
| Plus | Beads | | | | |
| HBSS | — | — | 1 X | — | |
| 1% Flow- | — | — | 1% | 1% | |
| Fix PFA | | | | | |

Table 6, and Table 7 and Table 9: Samples for bead size, compensation of the flow cytometer, isotype control, sputum background and treated sputum are prepared as described.

TABLE 6
Tubes for instrument settings

| | | Comp | Comp | | | | | | | | | |
|---|---|---|---|---|---|---|---|---|---|---|---|---|
| | | Bead | Bead | Anti- | Anti- | Anti- | Anti- | Anti- | Anti- | Anti- | | Total |
| Tube | | Plus | Plus | CD45 | CD66b | CD3 | CD19 | CK | CD206 | EpCAM | HBSS | Volume |
| # | Name | (+) | (−) | (μL) | (μL) | (μL) | (μL) | (μL) | (μL) | (μL) | (μL) | (μL) |
| 1 | PE - beads | 1 drop* | 1 drop | 4 | | | | | | | 76 | 200 |
| 2 | LEAVE BLANK | | | | | | | | | | | |
| 3 | PE-CF594 - beads | 1 drop | 1 drop | | | | | | 4 | 4 | 72 | 200 |

*1 drop = 60 μl

TABLE 7
Tubes for sample analysis

| | | Sputum | | | | Antibodies (μl) | | | |
|---|---|---|---|---|---|---|---|---|---|
| | | plug | Cell | | | | PE- | | |
| Tube | | weight | volume | HBSS | FVS510 | Anti- | CF594 | Alexa488- | FITC- |
| # | Name | (Step 4) | (μL) | (μL) | (μL) | CD45 | isotype | isotype | isotype |
| 4 | Unstained | Small | 20 | 80 | − | − | − | − | − |
| | | Medium | 50 | 50 | | | | | |
| | | Large | 50 | 50 | | | | | |
| 5 | Isotype | Small | 20 | 59.9 | 0.6 | 10 | 1.5 | 2 | 6 |
| | controls | Medium | 50 | 29.9 | 0.6 | | | | |
| | | Large | 50 | 29.5 | 1 | | | | |

| | | | | | | Anti- | Anti- | Anti- | Anti- | Anti- |
|---|---|---|---|---|---|---|---|---|---|---|
| | | | | | | CD45 | CD206 | CD3 | CD19 | CD66 |
| 6 | CyPath | Small | 115 | 92.25 | 1.5 | 25 | 3.75 | 2.5 | 2.5 | 7.5 |
| | Assay | Medium | 350 | 64.5 | 3 | 50 | 7.5 | 5 | 5 | 15 |
| | (Blood cells) | Large | 725 | 100 | 10 | 100 | 15 | 10 | 10 | 30 |

| | | | | | | Anti-CD45 | Anti-EpCAM | Anti-panCK |
|---|---|---|---|---|---|---|---|---|
| 7 | CyPath | Small | 115 | 101.5 | 1.5 | 25 | 5 | 2 |
| | Assay | Medium | 350 | 83 | 3 | 50 | 10 | 4 |
| | (Epithelial cells) | Large | 725 | 137 | 10 | 100 | 20 | 8 |

Tubes #1-#7 are incubated in the dark for 35 min. After antibody incubation, each tube is filled with cold HBSS, and the supernatant is spun down at 800×g for 10 minutes at 4° C. The supernatant is discarded and the pellet is resuspended as follows: To tubes #1-#3 add 0.5 mL cold HBSS to tubes and store on ice, at 4° C., until data acquisition by flow cytometry. To tube #4 and #5, add 2 mL cold 1% PFA fixative. To tubes #6 and #7 add 10 mL cold 1% PFA fixative. Incubate tubes for 1 hour on ice, covered with foil. After fixative incubation, fill each tube with cold HBSS. Spin down cells at 1600×g for 10 minutes at 4° C. Aspirate supernatant as much as possible, but without disturbing the pellet. Re-suspend the pellet in the residual fluid. Re-suspend tube #4 and #5 in 0.2 mL cold HBSS and store with tubes #1-#3 on ice, at 4° C., until data acquisition by flow cytometry. For tubes #6 and #7, add ice-cold HBSS according to the following formula:

Final volume (mL) of each tube=0.15*[Total Cell/10⁶] (formula 1)

For cell #, obtain cell count from a 1:40 diluted cell suspension with trypan blue. Add 10 μL of the 1:40 dilution to a hemocytometer and count the cells in all four large quadrants. Accurate cell count constitutes 25-60 cells per quadrant.

Place tubes #6 and #7 overnight on ice, at 4° C., until ready for TCPP labeling on day 2.

TABLE 9
TCPP labeling/instrument reagents

| Reagent | Company |
|---|---|
| HBSS | Gibco |
| 30 μm NIST beads | Polysciences |
| 20 μm NIST beads | Polysciences |
| 5 μm NIST beads | Polysciences |
| Rainbow beads | Spherotech |

CyPath Assay TCPP working solution is made as a 20 μg/mL TCPP solution (1:50 of stock), using cold HBSS and is protected from light. Obtain 1 tube with A549 cells to be used as unstained control for FVS and TCPP labeling (Tube #8). Obtain 1 tube with A549 cells to be used as compensation tube for FVS labeling (Tube #9). Obtain 1 tube with A549 cells to be used as compensation tube for TCPP labeling (Tube #10). Obtain 1 tube with A549 cells to be used as compensation tube for PanCK labeling (Tube #11)

### TCPP Labeling

Add Cypath Assay TCPP working solution volume according to Table 10

TABLE 10
TCPP labeling volumes

| Tube | | | Actual |
|---|---|---|---|
| # | Cells | Volume TCPP working solution | volume used |
| 6 | Sputum | Same volume as calculated for formula 1 | |
| 7 | Sputum | Same volume as calculated for formula 1 | |
| 10 | A549 | 300 μL | 300 μL |

Incubate the samples with TCPP for about 1 hour, fill tubes #6, #7 and 10 with cold HBSS and centrifuge at 1000×g for 15 minutes at 4 C. Aspirate supernatant without disturbing pellet. For tubes #6, #7 and #10 wash the pellet with cold HBSS and repeat centrifuge and wash steps. For tubes #6, #7 and #10 re-suspend the pellet in the residual fluid and add 300 μL cold HBSS to tube #10, if the total cell count is <20×10⁶ cells total, then add 250 μL of cold HBSS to tubes #6 and #7 to transfer the cells from the 15 mL conical tube to a flow cytometry tube (labeled #6 and #7, respectively).

Flow Cytometric Data Acquisition

The flow cytometry acquisition rate of 10,000 events/sec or lower is preferred with the following settings: Parameters used on the LSRII include: Threshold, FSC voltage, SSC voltage, BV510 voltage wherein this voltage should be checked on ALL cells, including the BECs, PE voltage, FITC voltage, PE-TxRed voltage, and APC voltage. For optimization of the assay using equivalent flow cytometers, one of ordinary skill in the art will know preferred settings to achieve same or similar results.

Summary of fluorescence intensity values that determine the population gates:

BLOOD: 6 gates

| Fluorophore | average | range |
|---|---|---|
| To set pop 1 | | |
| FITC | 600 | 200-1050 |
| PE-CF594 | 500 | 200-750 |
| To set pop 5 | | |
| FITC (boundary with pop 6) | 3300 | 1,000-6,000 |
| PE-CF594 (left boundary) | 13,000 | 8,000-20,000 |

EPITHELIAL: 9 gates

| Fluorophore | average | range |
|---|---|---|
| To set pop 1 | | |
| FITC | 250 | 90-500 |
| PE-CF594 | 450 | 100-1,000 |
| To set cut off for high expression | | |
| FITC | 14,200 | 10,000-30,000 |
| PE-CF594 | 3,000 | 1,000-6,000 |
| | | (=10-20 × value of the |
| | | PE-CF594 value that |
| | | determines pop 1) |

It should be noted that the referenced settings are specific for the LSRII instrument and may vary for other flow cytometers but will be apparent to one of ordinary skill in the rt how to compensate for the different instruments to produce comparable ranges.

While the above examples are exemplary for lung cancer detection, other diseases and conditions of the lung can be detected and/or monitored over time with a system and method as disclosed herein. For example, when the subject is suspected of developing or prone to have an exacerbation of symptoms associated with lung diseases such as asthma, COPD, influenza, chronic bronchitis, tuberculosis, cystic fibrosis, pneumonia, graft-vs.-host disease, sputum may be analyzed for the alterations in the distribution of cell populations as compared to a database of control (non-diseased) and disease sample profiles.

Note that in the specification and claims, “about” or “approximately” means within twenty percent (20%) of the numerical amount cited. All computer software disclosed herein may be embodied on any computer-readable medium (including combinations of mediums), including without limitation CD-ROMs, DVD-ROMs, hard drives (local or network storage device), USB keys, other removable drives, ROM and firmware.

In at least one embodiment, and as readily understood by one of ordinary skill in the art, the apparatus, according to the invention, will include a general- or specific-purpose computer or distributed system programmed with computer software implementing the steps described above, which computer software may be in any appropriate computer language, including C++, FORTRAN, BASIC, Java, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements. The multidimensional data recorded from the cells and particles analyzed as they move through the flow cytometer are recorded and permit analysis and fractionation of the cell populations based upon the multidimensional optical properties.

### REFERENCES

1. National Lung Screening Trial Research Team, Church T R, Black W C, Aberle D R, Berg C D, Clingan K L, et al. Results of initial low-dose computed tomographic screening for lung cancer. N Engl J Med. 2013 May 23; 368(21):1980-91.
2. Krantz S B, Meyers B F. Health risks from computed tomographic screening. Thorac Surg Clin. 2015 May; 25(2):155-60.
3. Sacher A G, Komatsubara K M, Oxnard G R. Application of Plasma Genotyping Technologies in Non-Small Cell Lung Cancer: A Practical Review. J Thorac Oncol. 2017 Sep. 1; 12(9):1344-56.
4. Li T, Kung H-J, Mack P C, Gandara D R. Genotyping and Genomic Profiling of Non-Small-Cell Lung Cancer: Implications for Current and Future Therapies. J Clin Oncol. 2013 Mar. 10; 31(8):1039-49.
5. The Clinical Lung Cancer Genome Project (CLCGP) and Network Genomic Medicine (NGM). A Genomics-Based Classification of Human Lung Tumors. Sci Transl Med. 2013 Oct. 30; 5(209):209ra153.
6. Speicher M R, Pantel K. Tumor signatures in the blood. Nat Biotechnol. 2014 May; 32(5):441-3.
7. Stahl D L, Richard K M, Papadimos T J. Complications of bronchoscopy: A concise synopsis. Int J Crit Illn Inj Sci. 2015; 5(3):189-95.
8. Thunnissen FBJM. Sputum examination for early detection of lung cancer. J Clin Pathol. 2003 November; 56(11):805-10.
9. Gao W, Keohavong P. Detection of point mutations of K-ras oncogene and p53 tumor-suppressor gene in sputum samples. Methods Mol Biol Clifton N.J. 2014; 1105:325-44.
10. Li G, Guillaud M, LeRiche J, McWilliams A, Gazdar A, Lam S, et al. Automated Sputum Cytometry for Detection of Intraepithelial Neoplasias in the Lung. Anal Cell Pathol Amst. 2012; 35(3):187-201.
11. Osterloh J, Vicente M. Mechanisms of porphyrinoid localization in tumors. J Porphyr Phthalocyaniones. 2002; 6:305-24.
12. Patriquin L, Merrick D T, Hill D, Holcomb R G, Lemieux M E, Bennett G, et al. Early Detection of Lung Cancer with Meso Tetra (4-Carboxyphenyl) Porphyrin-Labeled Sputum. J Thorac Oncol. 2015 Sep. 1; 10(9):1311-8.
13. Wood D E. National Comprehensive Cancer Network (NCCN) Clinical Practice Guidelines for Lung Cancer Screening. Thorac Surg Clin. 2015 May; 25(2):185-97.
14. Paszkiewicz G M, Timm E A, Mahoney M C, Wallace P K, Nasca M A S, Tammela T L, et al. Increased Human Buccal Cell Autofluorescence Is a Candidate Biomarker of Tobacco Smoking. Cancer Epidemiol Prev Biomark. 2008 Jan. 1; 17(1):239-44.
15. Pizzichini E, Pizzichini M M, Efthimiadis A, Hargreave F E, Dolovich J. Measurement of inflammatory indices in induced sputum: effects of selection of sputum to minimize salivary contamination. Eur Respir J. 1996 Jun. 1; 9(6):1174-80.
16. Thomas H. Sputum: preparation and examination of Gram stained smears [Internet]. Available from: https://www.uvmhealth.org/medcenter/Documents/8138Sputum_Gram_Stain.ppt
17. Franks T J, Colby T V, Travis W D, Tuder R M, Reynolds H Y, Brody A R, et al. Resident cellular components of the human lung: current knowledge and goals for research on cell phenotyping and function. Proc Am Thorac Soc. 2008 Sep. 15; 5(7):763-6.
18. Korbelik M, Krosl G, Chaplin D J. Photofrin Uptake by Murine Macrophages. Cancer Res. 1991 May 1; 51(9):2251-5.
19. Korbelik M, Krosl G, Olive P L, Chaplin D J. Distribution of Photofrin between tumour cells and tumour associated macrophages. Br J Cancer. 1991 September; 64(3):508-12.
20. Figge F H J, Weiland G S, Manganiello L O J. Cancer detection and therapy; affinity of neoplastic, embryonic, and traumatized tissues for porphyrins and metalloporphyrins. Proc Soc Exp Biol Med Soc Exp Biol Med N Y N. 1948 August; 68(3):640.
21. Rassmussen-Taxdal D S, Ward G E, Figge F H J. Fluorescence of human lymphatic and cancer tissues following high doses of intravenous hematoporphyrin. Cancer. 1955 Jan. 1; 8(1):78-81.
22. Altman K I, Salomon K. Localization of a halogenated porphyrin in mouse tumours. Nature. 1960 Sep. 24; 187:1124.
23. Schupp J, Krebs F K, Zimmer N, Trzeciak E, Schuppan D, Tuettenberg A. Targeting myeloid cells in the tumor sustaining microenvironment. Cell Immunol [Internet]. 2017 Nov. 1 [cited 2017 Nov. 30]; Available from: http://www.sciencedirect.com/science/article/pii/S0008874917301909
24. Ward-Hartstonge K A, Kemp R A. Regulatory T-cell heterogeneity and the cancer immune response. Clin Transl Immunol. 2017 Sep. 15; 6(9):e154.
25. Barnes T A, Amir E. HYPE or HOPE: the prognostic value of infiltrating immune cells in cancer. Br J Cancer. 2017 Aug. 8; 117(4):451-60.
26. Murray P J. Nonresolving macrophage-mediated inflammation in malignancy. FEBS J. :n/a-n/a.
27. Brooks C R, van Dalen C J, Hermans I F, Douwes J. Identifying leukocyte populations in fresh and cryopreserved sputum using flow cytometry. Cytometry B Clin Cytom. 2013 Mar. 1; 84B(2): 104-13.
28. Vidal S, Bellido-Casado J, Granel C, Crespo A, Plaza V, Juárez C. Flow cytometry analysis of leukocytes in induced sputum from asthmatic patients. Immunobiology. 2012 July; 217(7):692-7.
29. Leckie M J, Jenkins G R, Khan J, Smith S J, Walker C, Barnes P J, et al. Sputum T lymphocytes in asthma, COPD and healthy subjects have the phenotype of activated intraepithelial T cells (CD69+CD103+). Thorax. 2003 January; 58(1):23-9.
30. Shiota Y, Matsumoto H, Hiyama J, Okamura M, Ono T, Mashiba H. Flow cytometric analysis of lymphocytes and lymphocyte subpopulations in induced sputum from patients with asthma. Allergol Int. 2000; 49:125-33.
31. Papanicolaou Society of Cytopathology Task Force, on Standards of Practice. Guidelines of the Papanicolaou Society of Cytopathology for the examination of cytologic specimens obtained from the respiratory tract. Papanicolaou Society of Cytopathology Task Force on Standards of Practice. Diagn Cytopathol. 1999 July; 21(1):61-9.
32. Freeman C M, Crudgington S, Stolberg V R, Brown J P, Sonstein J, Alexis N E, et al. Design of a multi-center immunophenotyping analysis of peripheral blood, sputum and bronchoalveolar lavage fluid in the Subpopulations and Intermediate Outcome Measures in COPD Study (SPIROMICS). J Transl Med. 2015 Jan. 27; 13:19.
33. Yu Y-RA, Hotten D F, Malakhau Y, Volker E, Ghio A J, Noble P W, et al. Flow Cytometric Analysis of Myeloid Cells in Human Blood, Bronchoalveolar Lavage, and Lung Tissues. Am J Respir Cell Mol Biol. 2016 January; 54(1):13-24.
34. Desch A N, Gibbings S L, Goyal R, Kolde R, Bednarek J, Bruno T, et al. Flow Cytometric Analysis of Mononuclear Phagocytes in Nondiseased Human Lung and Lung-Draining Lymph Nodes. Am J Respir Crit Care Med. 2016 Mar. 15; 193(6):614-26.
35. Patel V I, Booth J L, Duggan E S, Cate S, White V L, Hutchings D, et al. Transcriptional Classification and Functional Characterization of Human Airway Macrophage and Dendritic Cell Subsets. J Immunol. 2017 Feb. 1; 198(3):1183-201.
36. Baharom F, Rankin G, Blomberg A, Smed-Sörensen A. Human Lung Mononuclear Phagocytes in Health and Disease. Front Immunol [Internet]. 2017 May 1 [cited 2017 Dec. 13]; 8. Available from: https://www.ncbi.nlm.nih.gov/pmc/articles/PMC5410584/37.
37. Lay J C, Peden D B, Alexis N E. Flow cytometry of sputum: assessing inflammation and immune response elements in the bronchial airways. Inhal Toxicol. 2011 June; 23(7):392-406.
38. Erozan Y S, Frost J K. Cytopathologic diagnosis of cancer in pulmonary material: a critical histopathologic correlation. Acta Cytol. 1970 December; 14(9):560-5.
39. Hinson K F, Kuper S W. THE DIAGNOSIS OF LUNG CANCER BY EXAMINATION OF SPUTUM. Thorax. 1963 December; 18:350-3.
40. Johnston W W, Bossen E H. Ten years of respiratory cytopathology at Duke University Medical Center. I. The cytopathologic diagnosis of lung cancer during the years 1970 to 1974, noting the significance of specimen number and type. Acta Cytol. 1981 April; 25(2):103-7.
41. Farber S M. Clinical appraisal of pulmonary cytology. JAMA. 1961 Feb. 4; 175:345-8.
42. Ng A B, Horak G C. Factors significant in the diagnostic accuracy of lung cytology in bronchial washing and sputum samples. II. Sputum samples. Acta Cytol. 1983 August; 27(4):397-402.
43. Wheater P R, Burkitt H G, Daniels V G. Functional histology. first edition. Norwich, England: Jarrold & Sons Ltd; 1979.
44. Krombach F, Munzing S, Allmeling A M, Gerlach J T, Behr J, Dorger M. Cell size of alveolar macrophages: an interspecies comparison. Environ Health Perspect. 1997 September; 105 Suppl 5:1261-3.
45. Kini S R. Color atlas of pulmonary cytopathology. Springer-Verlag New York, INc.; 2002.301 p.
46. Fillmore C M, Xu C, Desai P T, Berry J M, Rowbotham S P, Lin Y-J, et al. EZH2 inhibition sensitizes BRG1 and EGFR mutant lung tumors to Topoll inhibitors. Nature. 2015 Apr. 9; 520(7546):239-42.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A method of predicting the likelihood of lung disease in a subject, said method comprising the steps of:
labeling an ex-vivo sputum sample with one or more of the following:
i) a first labeled probe that binds a biomarker expressed on a white blood cell population of sputum cells;
ii) a second labeled probe selected from the group consisting of: a granulocyte probe that binds a biomarker expressed on a granulocyte cell population of sputum cells, a T-cell probe that binds a biomarker expressed on a T-cell cell population of sputum cells, a B-cell probe that binds a biomarker expressed on a B-cell cell population of sputum cells, or any combination thereof;
iii) a third labeled probe that binds a biomarker on a macrophage cell population;
iv) a fourth labeled probe that binds to a disease related cell in the sputum sample;
v) a fifth labeled probe that binds to a biomarker expressed on an epithelial cell population of sputum cells; and
vi) a sixth labeled probe that binds to a cell surface biomarker expressed on an epithelial cell population of sputum cells;
flow cytometrically analyzing the labelled sputum sample to obtain data comprising per cell cytometric data based upon a mean fluorescent signature of any of the i)-vi) labeled probes; and
detecting from the per cell data the likelihood of lung disease in a subject based upon a profile of a presence or absence of labeled probes in the per cell labelled data.

2. The method of claim 1 further comprising determining a ratio of the sputum cells in the data collected from the labelled sputum sample that are negative for i) as compared to the sputum cells that are positive for i) to identify a biomarker 1.

3. The method of claim 2 wherein the ratio of less than 2 indicates the sputum sample is positive for biomarker 1.

4. The method of claim 3 wherein the positive biomarker 1 has a sensitivity of at least about 80% and a specificity of at least 50%.

5. The method of claim 1 further comprising determining from the data collected from the labeled sputum sample the sputum cells that are negative for i) and positive for iv) and v) to identify a biomarker 2.

6. The method of claim 5 wherein a percentage of sputum cells negative for i) and positive for iv) and v) that is greater than 0.03% indicates the sputum sample is positive for biomarker 2.

7. The method of claim 6 wherein the positive biomarker 2 has a sensitivity of at least 90% and a specificity of at least 50%.

8. The method of claim 3 further comprising determining from the data collected from the labeled sputum sample the sputum cells that are negative for i) and positive for iv) and v) to identify a biomarker 2.

9. The method of claim 8 wherein a percentage of sputum cells negative for i) and positive for iv) and v) that is greater than 0.03% indicates the sputum sample is positive for biomarker 2.

10. The method of claim 9 wherein a combination of the positive biomarker 1 and the positive biomarker 2 have a sensitivity of at least 80% and a specificity of at least 80%.

11. The method of claim 1 further comprising determining from the data collected from the labeled sputum sample the sputum cells that are positive for i), iii) and display FITC autofluorescence to identify a biomarker 3.

12. The method of claim 11 wherein a percentage of sputum cells positive for i), iii) and display FITC autofluorescence that is greater than 0.03% indicates the sputum sample is positive for biomarker 3.

13. The method of claim 12 wherein the positive biomarker 3 has a sensitivity of at least 60% and a specificity of at least 70%.

14. The method of claim 9 further comprising determining from the data collected from the labeled sputum sample the sputum cells that are positive for i), iii) and v) to identify a biomarker 3.

15. The method of claim 14 wherein a percentage of sputum cells positive for i), iii) and display FITC autofluorescence that is greater than 0.03% indicates the sputum sample is positive for biomarker 3.

16. The method of claim 15 wherein the combination of the positive biomarkers 1, 2, and 3 have a sensitivity of at least 80% and a specificity of at least 80%.

17. The method of claim 1 further comprising determining from the data collected from the labeled sputum sample the sputum cells that are negative for i) and positive for v) and vi) to identify a biomarker 4.

18. The method of claim 17 wherein the percentage of cells negative for i) and positive for v) and vi) more than 2% indicates the sample is positive for biomarker 4.

19. The method of claim 18 wherein the positive biomarker 4 has a sensitivity of at least 70% and a specificity of at least 70%.

20. The method of claim 15 further comprising determining from the data collected from the labeled sputum sample the sputum cells that are negative for i) and positive for v) and vi) to identify a biomarker 4.

21. The method of claim 20 wherein a percentage of cells negative for i) and positive for v) and vi) of more than 2% indicates the sample is positive for biomarker 4.

22. The method of claim 21 wherein the combination of the positive biomarkers 1, 2, 3 and 4 have a sensitivity of at least 70% and a specificity of at least 75%.

23. The method of claim 1 wherein the flow cytometric analysis comprises excluding from data analysis those cells that have a diameter of less than about 5 μm and greater than about 30 μm.

24. The method of claim 1 wherein the flow cytometric analysis comprises excluding from data analysis those cells that are dead cells and cell clumps of more than one.

25. The method of claim 1 wherein the first labeled probe that binds a biomarker expressed on a white blood cell population of sputum cells is CD45 antibody or fragment thereof.

26. The method of claim 1 wherein the second labeled probe is the granulocyte probe that binds a biomarker expressed on a granulocyte cell population of sputum cells is a CD66b antibody or fragment thereof.

27. The method of claim 1 wherein the second labeled probe is the T-cell probe that binds a biomarker expressed on a T-cell cell population of sputum cells is a CD3 antibody or fragment thereof.

28. The method of claim 1 wherein the second labeled probe is the B-cell probe that binds a biomarker expressed on a B-cell cell population of sputum cells is a CD19 antibody or fragment thereof.

29. The method of claim 1 wherein the second labeled probe is a combination of the granulocyte probe, the T-cell probe, and the B-cell probe.

30. The method of claim 29 wherein the granulocyte probe is a CD66b antibody or fragment thereof, the T-cell probe is a CD3 antibody or fragment thereof and the B-cell probe is a CD19 antibody or fragment thereof.

31. The method of claim 1 wherein the third labeled probe that binds a biomarker on a macrophage cell population of sputum cells is a CD206 antibody or fragment thereof.

32. The method of claim 1 wherein the fourth labeled probe that binds to a disease related cell in the sputum sample is a tetra (4-carboxyphenyl) porphyrin (TCPP).

33. The method of claim 1 wherein the fifth labeled probe that binds to a biomarker expressed on an epithelial cell population of sputum cells is a panCytokeratin antibody or fragment thereof.

34. The method of claim 1 wherein the sixth labeled probe that binds to a cell surface biomarker expressed on an epithelial cell population of sputum cells is an EpCam antibody or fragment thereof.

35. The method of claim 1 wherein the disease related cells are lung cancer cells or tumor associated immune cells.

36. The method of claim 1 wherein the lung disease is selected from the group consisting of asthma, COPD, influenza, chronic bronchitis, tuberculosis, cystic fibrosis, pneumonia, graft vs. hose disease and lung cancer.

37. The method of claim 1 wherein the sputum cells are fixed or non-fixed.

38. The method of claim 1 wherein the data comprising per cell cytometric data based upon a mean fluorescent signature of any of the i)-vi) labeled probes produces a sputum sample signature.

39. The method of claim 38 wherein the sputum sample signature identifies the lung disease.

40. The method of claim 39 wherein the lung disease is selected from the group consisting of asthma, COPD, influenza, chronic bronchitis, tuberculosis, cystic fibrosis, pneumonia, graft vs. hose disease and lung cancer.

41. The method of claim 39 wherein the sputum sample signature is compared to a database of control sputum sample signatures (non-diseased) and lung disease sample signatures to identify lung disease.

42. A first reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome; and a fluorochrome-conjugated antibodies or fragments thereof directed against cell's markers selected from; ii) EpCAM, and/or panCytokeratin, iii) CD45, CD206, CD3, CD19, CD66b or any combination thereof.

43. A second reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome and fluorochrome-conjugated antibodies or fragments thereof directed against the following cell's markers; ii) EpCAM and/or panCytokeratin, and iii) CD45.

44. A third reagent composition for flow cytometric phenotyping of sputum cells from a sputum sample of a subject to identify one or more biomarkers within the population of cells that are associated with a likelihood of lung disease, wherein the reagent composition comprises: i) a tetra (4-carboxyphenyl) porphyrin (TCPP) fluorochrome; and fluorochrome-conjugated antibodies or fragments thereof directed against one or more of the following cell's markers; CD45, CD206, CD3, CD19, and CD66b.

45. A method of predicting the likelihood of lung disease in a subject, said method comprising the steps of:
labeling an ex-vivo sputum sample with i) a labeled probe that binds to a disease related cell in the sputum sample and ii) one or more fluorochrome-conjugated probes directed against a sputum cell's markers; and
flow cytometrically analyzing the labelled sputum sample to obtain data comprising per cell cytometric data based upon a mean fluorescent signature of any of the i)-ii) labeled probes; and
detecting from the per cell data the likelihood of lung disease in a subject based upon a profile of a presence or absence of i) and ii) in the per cell labelled data.

46. The method of claim 45 wherein the data comprising per cell cytometric data based upon a mean fluorescent signature of any of the i)-ii) produces a sputum sample signature.

47. The method of claim 46 wherein the sputum sample signature identifies the lung disease.

48. The method of claim 47 wherein the lung disease is selected from the group consisting of asthma, COPD, influenza, chronic bronchitis, tuberculosis, cystic fibrosis, pneumonia, graft vs. hose disease and lung cancer.

49. The method of claim 46 wherein the sputum sample signature is compared to a database of control sputum sample signatures (non-diseased) and lung disease sample signatures to identify the lung disease.

50. The method of claim 45 wherein the labeled probe that binds to the disease related cell in the sputum sample is a tetra (4-carboxyphenyl) porphyrin (TCPP).
